(19) United States
(12) Patent Application Publication
(10) Pub. No.: US 2011/0106012 A1
(43) Pub. Date: May 5, 2011

(54) Sheath Introducer with Self-Anchoring Mechanism

(76) Inventor: Franz E. Velarde, Harlingen, TX (US)

(21) Appl. No.: 12/915,543

(22) Filed: Oct. 29, 2010

Related U.S. Application Data
(60) Provisional application No. 61/256,162, filed on Oct. 29, 2009.

(51) Int. Cl. A61M 25/09; A61M 25/00

(52) U.S. Cl. 604/164.03

(57) ABSTRACT

An assembly for accessing a vessel comprises an elongate housing having a longitudinal axis, a proximal end, and a distal end opposite the proximal end. The housing includes a base extending from the proximal end, an elongated tubular extending from the distal end to the base, and a throughbore extending axially from the proximal end to the distal end. In addition, the assembly comprises a stylet extending axially through the throughbore of the housing. The stylet has a proximal end comprising an actuation head external the throughbore of the housing, and a distal end opposite the proximal end of the stylet. Further, the assembly comprises an expandable member coupled to the distal end of the housing or the distal end of the stylet. The expandable member has an expanded position with a first diameter and an unexpanded position with a second diameter that is less than the first diameter. Further, the stylet is adapted to transition the expandable member between the unexpanded position and the unexpanded position by moving axially through the housing.

### CROSS-REFERENCE TO RELATED APPLICATIONS

This application claims benefit of U.S. provisional patent application Ser. No. 61/256,162 filed Oct. 29, 2009, and entitled Sheath Introducer with Self-Anchoring Mechanism,” which is hereby incorporated herein by reference in its entirety.

### STATEMENT REGARDING FEDERALLY SPONSORED RESEARCH OR DEVELOPMENT

Not applicable.

### BACKGROUND

1. Field of the Invention

The present invention relates to the fields of vascular surgery and interventional radiology. More particularly, the invention relates to a method and apparatus for the reconstitution of flow within a vascular conduit. The invention further relates to treatment of thrombosed and stenotic hemodialysis access (i.e., arteriovenous grafts and fistulas).

2. Background of the Technology

Kidneys function to cleanse the blood by removing excess fluid, minerals, and wastes such as such as creatinine and urea. They also make hormones important for bone strong and blood health. Among other problems, when kidneys fail, harmful wastes begin to build up within the body, blood pressure may rise, and excess fluid may be retained. Hemodialysis is a common method used to treat advanced and permanent kidney failure. Specifically, hemodialysis removes waste products such as creatinine and urea, as well as free water from the blood when the kidneys are in renal failure. Hemodialysis accesses to the patient's circulatory system, and allows blood to flow through a special filter that removes wastes and excess fluids. The cleansed blood is then returned to the body.

In hemodialysis, three primary methods are used to gain access to the blood: an arteriovenous (AV) fistula, a synthetic graft, or an intravenous catheter. In the United States, fistulas and grafts are employed in about 80% of hemodialysis procedures, and catheters are used about 20% of the time. As shown in FIG. 1, to create an arteriovenous (AV) fistula 10, a vascular surgeon joins an artery 11 and a vein 12 together through anastomosis (i.e., connection of artery 11 and vein 12). Since this bypasses the capillaries downstream of artery 11 and upstream of vein 12, blood flows relatively rapidly through fistula 10. Fistulas are usually created in the nondominant arm and may be situated on the hand, the forearm, or the elbow. After the anastomosis, the fistula will typically take a few weeks to heal and mature. During subsequent hemodialysis treatments, a first needle 13 and a second needle 14 downstream of the first needle 14 are inserted into fistula 10—first needle 13 draws blood from the circulatory system and passes it through the dialysis machine, and second needle 14 returns the cleansed blood from the dialysis machine back to the circulatory system. Some advantages of AV fistulas include lower infection rates since no foreign material is involved in their formation, higher blood flow rates (which translates to more effective dialysis), and a lower incidence of thrombosis.

Referring now to FIG. 2, to create an arteriovenous (AV) graft 20, an artificial vessel 21 is used to joint artery 11 with vein 12. Thus, AV grafts are similar to fistulas, except that an artificial vessel is used to join the artery and vein. Artificial vessel 21 is usually is made of a synthetic material, often PTFE, but sometimes chemically treated, sterilized veins from animals are used. Grafts are often employed when the patient's native vasculature does not permit a fistula. Grafts typically mature faster than fistulas, however, grafts are more prone to recurrent stenosis (narrowing) and thrombosis (clotting), particularly where the graft has been sewn to the vein and artery. Further, since grafts introduce foreign material to the body, they generally present a greater risk of infection. Since grafts can be made quite long, more sites on the body are candidates for grafts.

The mean problem-free patency period after creation after formation of an AV fistula is about 3 years, and the mean problem-free patency period after creation of an AV graft is about 1-2 years. The most common limitations to extended patency periods with AV fistulas and grafts are recurrent stenosis and thrombosis. Consequently, failing, stenosed, and thrombosed AV fistulas and grafts require repeated angioplasty to widen the stenosed vessels and/or thrombectomy to remove clots in the vessels.

All dialysis accesses (e.g., AV fistulas and grafts) eventually fail, and a new access has to be created. Further, there are a limited number of veins that can be used for access creation. Thus, some patients eventually run out of veins and options for dialysis access. If a kidney is not available for transplantation by the time all available accesses have been utilized, the patient is highly likely to succumb to renal failure. Therefore, it is paramount to treat failing dialysis accesses, in order to keep them functional for as long as possible.

In the majority of cases, the lesion that has caused the access failure is positioned in the venous limb. However, in many cases, it is not clear which limb is the arterial limb and which is the venous limb. In particular, every patient and every access (e.g., AV graft or AV fistula) is different. Further, many patients do not know or remember which limb of an access is the venous limb. Moreover, due to occlusion of the access and no flow therethrough, the physician may also be unable to easily determine which limb is the venous limb. Consequently, in most cases, both the arterial limb and the venous limb are accessed and treated. The conventional approach to perform a thrombectomy (i.e., declot) and angioplasty (i.e., widening) on an AV graft or AV fistula is to utilize two punctures—a vascular sheath extends through one puncture and provides access to the downstream venous limb of the graft/fistula, and a second vascular sheath extends through the second puncture and provides access to the upstream arterial limb of the graft/fistula. For example, as shown in FIG. 3, a first puncture 30 and a second puncture 31 provide access to AV graft 20. A first vascular sheath 32 is advanced through first puncture 30 into AV graft 20, and provides access to the venous limb 20b. With first sheath 32 properly positioned, mechanical or chemical thrombectomy is performed, followed by balloon and/or stent angioplasty. Venogram is then performed to confirm that all stenosed segments have been successfully treated and that conduit/vein caliber has been restored. Attention is then turned to the arterial limb 20a of the AV graft 20. A second vascular sheath 33 is advanced through second puncture 31 into AV graft 20, and provides access to the arterial limb 20a. As a result, sheaths 32, 33 are disposed in a crisscrossed arrangement. With first sheath 33 properly positioned, mechanical or chemical thrombectomy is performed, followed by balloon and/or stent angioplasty. Venogram is then performed to confirm that all stenosed segments have been successfully treated and that conduit/vein caliber has been restored.

Although this conventional approach provides the desired access to both the arterial and venous limbs of the graft/fistula, it has drawbacks. For instance, overlapping, crisscrossed vascular sheaths are cumbersome to position and handle, and further, often requires increased radiation exposure to the patient and operators due to positioning and manipulation of multiple instruments. The simultaneous positioning and manipulation of two vascular sheaths through two punctures also increase patient discomfort and risks for bleeding and infection. In addition, “dead space” in the AV graft/fistula between the vascular sheaths may inhibit the ability to completely remove clots. Still further, placement of two vascular sheaths typically requires more equipment and is more time-consuming, making the procedure more expensive. Also, use of two needle punctures effectively doubles the potential risk of an accidental needle puncture to an operators and risk of transmission of blood borne diseases, such as Hepatitis and HIV. It should also be appreciated that every puncture site is followed by scarring, which is known to be associated with further dialysis access stenosis at puncture sites. Thus, minimizing the number of puncture sites should help prolong the life of the dialysis access.

Single puncture techniques to access AV grafts and fistulas for treatment of stenosis and thrombosis would reduce the likelihood of many of the aforementioned problems. However, single puncture access has not gained popularity, primarily due to difficulty in maintaining the sheath within the lumen of the graft or fistula while the sheath is being repositioned between the arterial limb and venous limb of the AV graft/fistula. Specifically, in clinical practice, it is often necessary to reposition a vascular sheath multiple times. However, in repositioning the vascular sheath, the tip of the vascular sheath may be inadvertently pulled and removed from the AV graft/fistula and access lost.

Empirical data suggests that even experienced operators lose access during repositioning of the vascular sheath in about 20% of the cases. Once access is lost, regaining access through the single puncture can be difficult. For example, if access is lost after arterial clot removal and heparinization, significant bleeding, with or without hematoma formation, may be experienced. In most cases, unsuccessful attempts to regain access will be followed by formation of a new puncture, thereby defeating the purpose of the single puncture technique. A second puncture may also increase the duration of the procedure since hemostasis must be achieved before the procedure can be restarted.

Accordingly, there remains a need in the art for improved systems, devices, and methods for accessing AV grafts and fistulas for the treatment of stenosis and thrombosis. Such systems, devices, and methods would be particularly well-received if they utilized a single access puncture and reduced likelihood of access loss during repositioning of the vascular sheath.

### BRIEF SUMMARY OF THE DISCLOSURE

These and other needs in the art are addressed in one embodiment by an assembly for accessing a vessel. In an embodiment, the assembly comprises an elongate housing having a longitudinal axis, a proximal end, and a distal end opposite the proximal end. The housing includes a base extending from the proximal end, an elongated tubular extending from the distal end to the base, and a throughbore extending axially from the proximal end to the distal end. In addition, the assembly comprises a stylet extending axially through the throughbore of the housing. The stylet has a proximal end comprising an actuation head external the throughbore of the housing, and a distal end opposite the proximal end of the stylet. Further, the assembly comprises an expandable member coupled to the distal end of the housing or the distal end of the stylet. The expandable member has an expanded position with a first diameter and an unexpanded position with a second diameter that is less than the first diameter. Further, the stylet is adapted to transition the expandable member between the unexpanded position and the unexpanded position by moving axially through the housing.

These and other needs in the art are addressed in another embodiment by a method for accessing a first limb and a second limb of an AV graft or fistula of a patient. In an embodiment, the method comprises (a) providing a vascular sheath having a distal end disposed in the first limb and a proximal end external the patient. The vascular sheath extends through a single puncture in the patient. In addition, the method comprises (b) inserting an introducer into the vascular sheath and advancing the introducer through the vascular sheath until a distal end of the introducer is disposed within the first limb. Further, the method comprises (c) radially expanding an expandable member of the introducer within the first limb. Still further, the method comprises (d) withdrawing the vascular sheath from the first limb through the single puncture with the introducer after (c). Moreover, the method comprises (e) restricting the distal end of the introducer from exiting the AV graft or fistula with the expandable member during (d). The method also comprises (f) advancing the introducer and the sheath through the single puncture and into the second limb.

Thus, embodiments described herein comprise a combination of features and advantages intended to address various shortcomings associated with certain prior devices, systems, and methods. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 is a schematic view of a an AV fistula created in the forearm of a patient;

FIG. 2 is a schematic view of an AV graft created in the forearm of a patient;

FIG. 3 is a schematic view of a conventional method for treating stenosis and thrombosis associated with an AV graft;

FIG. 4 is a side view of an embodiment of a vascular sheath;

FIG. 5 is a cross-sectional view of the vascular sheath of FIG. 4;

FIG. 6 is a side view of an embodiment of a vascular sheath introducer in accordance with the principles described herein;

FIG. 7 is a cross-sectional view of the introducer of FIG. 6;

FIG. 8 is a side view of the introducer of FIG. 6 with the expandable member in the expanded position;

FIG. 9 is a proximal end view of the introducer of FIG. 6;

FIGS. 10A and 10B are enlarged partial side views of the radially expandable member of FIG. 6 in an unexpanded position and a radially expanded position, respectively;

FIGS. 11A and 11B are enlarged partial side views of an embodiment of an expandable member in an unexpanded position and a radially expanded position, respectively;

FIGS. 12 and 13 are side views of the vascular sheath of FIG. 4 mounted to the introducer of FIG. 6 with the expandable member in the unexpanded position and the expanded position, respectively;

FIGS. 14-20 are schematic views of the introducer of FIG. 6 being employed to percutaneously reposition the vascular sheath of FIG. 4 from a venous limb of an AV graft to an arterial limb of an AV graft through a single puncture without losing access;

FIG. 21 is a cross-sectional view of an embodiment of a vascular sheath introducer in accordance with the principles described herein;

FIG. 22 is an enlarged partial cross-sectional view of the introducer of FIG. 21;

FIGS. 23-27 are schematic views of the introducer of FIG. 21 being employed to percutaneously position the vascular sheath of FIG. 4 in a venous limb of an AV graft, and then percutaneously reposition the vascular sheath of FIG. 4 from a venous limb of an AV graft to an arterial limb of an AV graft through a single puncture without losing access;

FIG. 28 is a side view of an embodiment of a vascular sheath introducer in accordance with the principles described herein;

FIG. 29 is a cross-sectional view of the introducer of FIG. 28;

FIG. 30 is a side view of the introducer of FIG. 28 with the expandable member in the unexpanded position;

FIGS. 31A and 31B are enlarged partial cross-sectional views of the expandable member of FIG. 28 in the relaxed, unexpanded position and the radially expanded position, respectively;

FIG. 32 is a proximal end view of the introducer of FIG. 28;

FIGS. 33 and 34 are side views of the vascular sheath of FIG. 4 mounted to the introducer of FIG. 28;

FIGS. 35-41 are schematic views of the introducer of FIG. 24 being employed to percutaneously reposition the vascular sheath of FIG. 4 from a venous limb of an AV graft to an arterial limb of an AV graft through a single puncture without losing access;

FIG. 42 is a cross-sectional view of an embodiment of a vascular sheath introducer in accordance with the principles described herein;

FIG. 43 is an enlarged partial cross-sectional view of the introducer of FIG. 42;

FIGS. 44-48 are schematic views of the introducer of FIG. 42 being employed to percutaneously reposition the vascular sheath of FIG. 4 from a venous limb of an AV graft to an arterial limb of an AV graft through a single puncture without losing access;

FIG. 49 is a side view of an embodiment of a vascular sheath introducer in accordance with the principles described herein;

FIG. 50 is a cross-sectional view of the introducer of FIG. 49;

FIGS. 51-53 are side views of the vascular sheath of FIG. 4 mounted to the introducer of FIG. 49;

FIGS. 54A, B, and C are enlarged partial cross-sectional views of the expandable member of FIG. 49 in an unexpanded position within the sheath of FIG. 4, an expanded position extending from the sheath of FIG. 4, and an unexpanded position within the sheath of FIG. 4, respectively;

FIG. 55 is a proximal end view of the introducer of FIG. 49;

FIGS. 56-63 are schematic views of the introducer of FIG. 49 being employed to percutaneously reposition the vascular sheath of FIG. 4 from a venous limb of an AV graft to an arterial limb of an AV graft through a single puncture without losing access;

FIG. 64 is a side view of an embodiment of a vascular sheath introducer in accordance with the principles described herein;

FIG. 65 is a cross-sectional view of the introducer of FIG. 64;

FIG. 66 is a side view of the introducer of FIG. 64 with the expandable member in the expanded position;

FIGS. 67A and 67B are distal end view of the introducer of FIG. 64 with the expandable member in the retracted, unexpanded position and the expanded position, respectively;

FIGS. 68 and 69 are side views of the vascular sheath of FIG. 4 mounted to the introducer of FIG. 64;

FIG. 70 is a side view of an embodiment of a vascular sheath introducer in accordance with the principles described herein;

FIG. 71 is a cross-sectional view of the introducer of FIG. 70;

FIGS. 72A and 72B are distal end view of the introducer of FIG. 70 with the expandable member in the retracted, unexpanded position and the expanded position, respectively;

FIG. 73 is a cross-sectional view of an embodiment of a vascular sheath introducer in accordance with the principles described herein; and

FIG. 74 is an enlarged partial view of the introducer of FIG. 73.

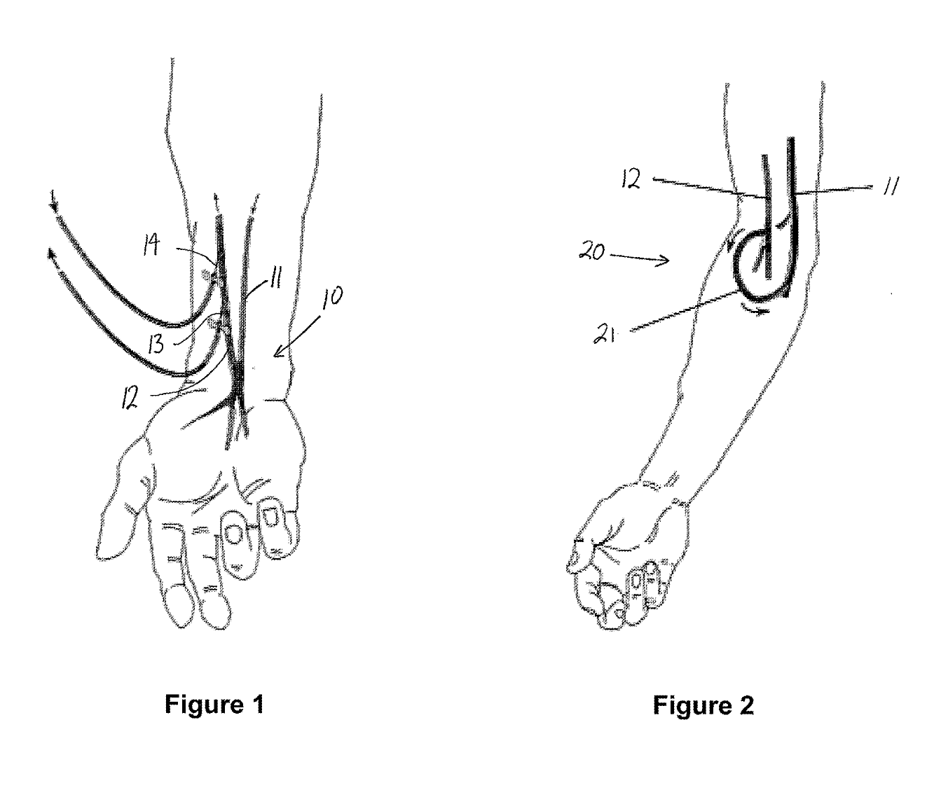

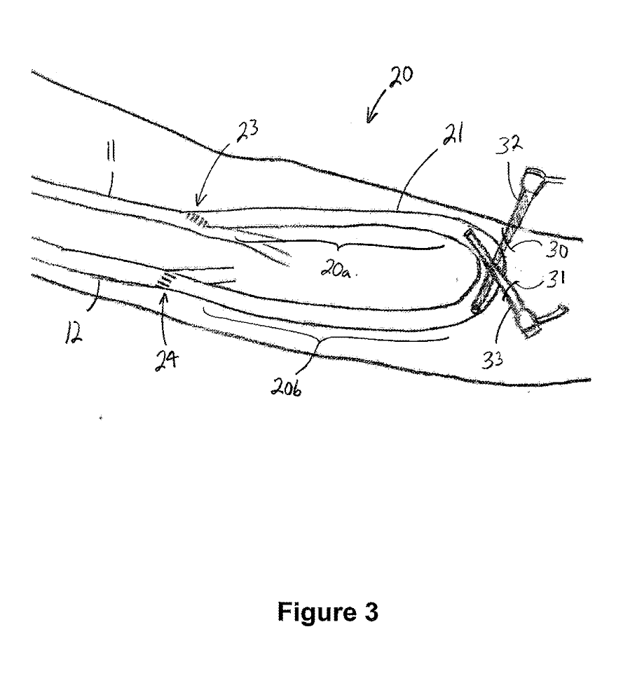

### DETAILED DESCRIPTION OF SOME OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

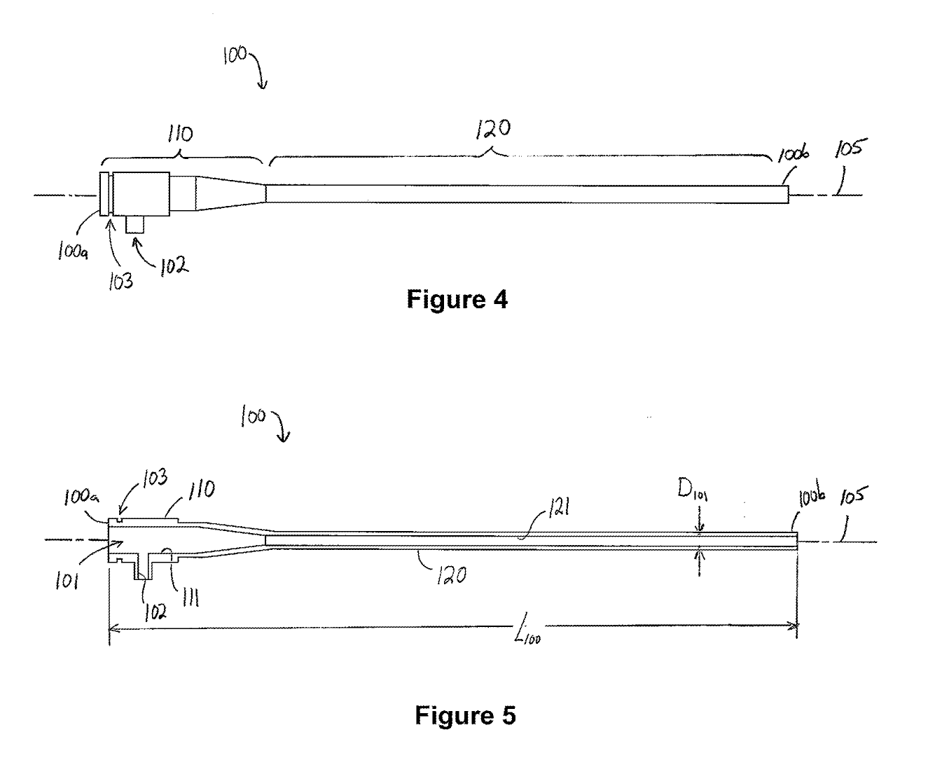

Referring now to FIGS. 4 and 5, an embodiment of a vascular sheath 100 for providing access to a vessel or lumen, AV graft, or AV fistula is shown. Sheath 100 has a central or longitudinal axis 105, a first or proximal end 100a, and a second or distal end 100b opposite end 100a. Sheath 100 has a length L₁₀₀ measured axially between ends 100a, b. In addition, sheath 100 comprises a base or body 110 and an elongate vascular access sleeve or tubular 120 extending axially from body 110. In particular, body 110 extends from first end 100a to access tubular 120, and access tubular 120 extends from second end 100b to body 110. In this embodiment, the radially outer surface of body 110 includes an annular recess 103 proximal first end 100a. Further, body 110 includes an axial throughbore or passage 111 and access tubular 120 includes an axial throughbore or passage 121 in fluid communication with body throughbore 111. Together, throughbores 111, 121 define an access throughbore 101 extending axially between ends 100a, b. Throughbore 101 has a minimum diameter D₁₀₁ defined by the inner diameter of tubular throughbore 121. A through port 102 extends radially through body 110 to access throughbore 101. Thus, port 102 is in fluid communication with access throughbore 101.

During stenosis or thrombosis treatments of AV grafts and fistula, vascular sheath 100 provides percutaneous access to the arterial limb and/or venous limb of the AV graft or fistula. As will be described in more detail below, vascular sheath 100 is passed through a puncture in the patient and into the AV graft or fistula to be treated. In particular, vascular sheath 100 is positioned with access tubular 120 extending through the puncture such that distal end 100b is disposed within the AV graft or fistula and body 110 is external the patient. Once sufficiently positioned, vascular sheath 100 allows instruments and/or medicine to be passed through access throughbore 101 and into the AV graft or fistula. In addition, port 102 allows access to throughbore 101 and AV graft or fistula. Once properly positioned within the AV graft or fistula, suitable tools including, without limitation, guiding wires, catheters, angioplasty balloons and/or stents, mechanical thrombectomy devices are passed through bore 101 to the portion of the AV graft or fistula to be treated. Further, port 102 is utilized to administer medications including, without limitation, anticoagulants, thrombolytic agents, antibiotics, and contrast material, and may also be used to aspirate blood or blood clots.

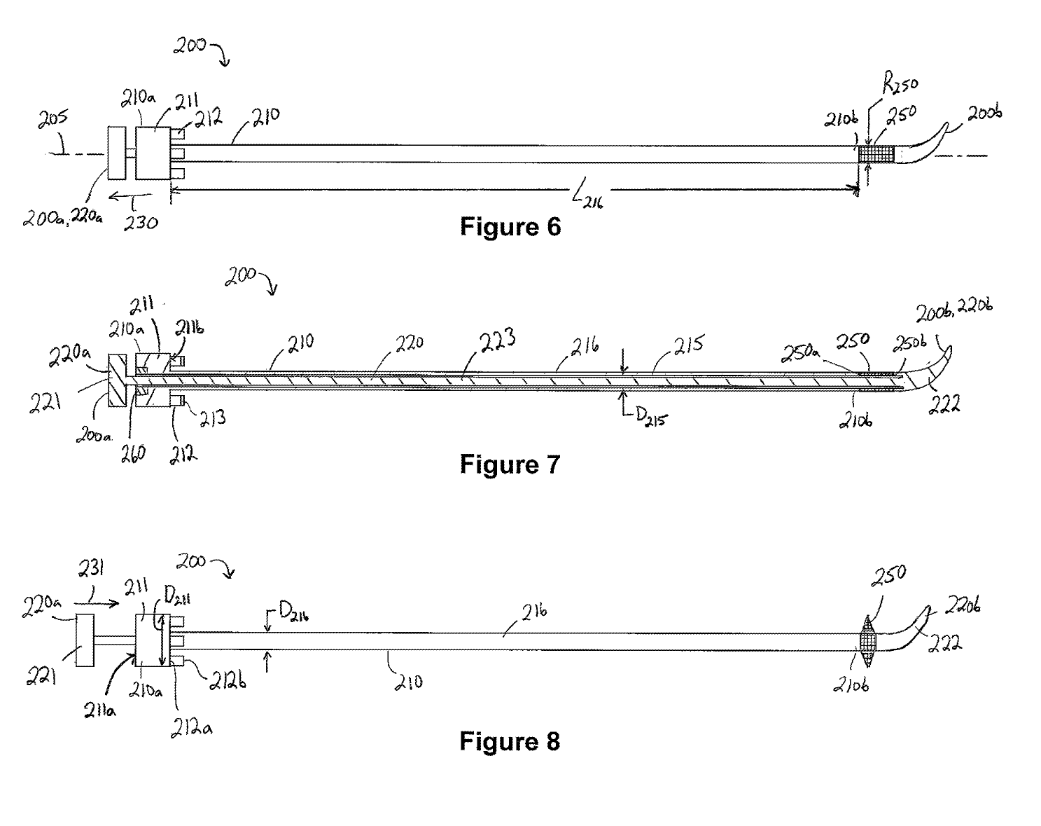

Referring now to FIGS. 6-9, an embodiment of a vascular sheath introducer 200 is shown. In general, introducer 200 is a device used to position a vascular sheath, such as vascular sheath 100 previously described, into a vessel, AV graft, or AV fistula, and manipulate the vascular sheath within the vessel, AV graft, or AV fistula. As will be described in more detail below, introducer 200 enables a vascular sheath (e.g., vascular sheath 100) to be pivoted or swiveled back and forth within the lumen of an AV graft or fistula between the venous limb and arterial limb as many times as necessary, while reducing likelihood of inadvertent access loss and associated problems. As a result, embodiments of introducer 200 allow the operator to access both the arterial and venous limb of an AV graft or fistula via a single puncture. Further, embodiments of introducer 200 allow the operator to access an AV graft or AV fistula at any level and work in antegrade and retrograde fashions, via a single puncture.

In this embodiment, introducer 200 has a central or longitudinal axis 205, a first or proximal end 200a, and a second or distal end 200b. In addition, introducer 200 includes a radially outer housing 210, a radially inner stylet 220 extending through housing 210, and a distal, radially expandable member 250 disposed about stylet 220 proximal end 200b. Housing 210, stylet 220, and expandable member 250 are each coaxially aligned with longitudinal axis 205. Further, stylet 220 is adapted to move axially relative to housing 210.

Referring still to FIGS. 6-9, housing 210 extends axially between a first or proximal end 210a and a second or distal end 210b. In addition, housing 210 includes a base 211 at proximal end 210a and an elongate tubular or sleeve 216 extending from base 211 to end 210b. Sleeve 216 has a length L₂₁₆ measured axially between base 211 and end 210b that is greater than vascular sheath length L₁₀₀. In this embodiment, base 211 and sleeve 216 are each generally cylindrical. In particular, base 211 has an outer diameter D₂₁₁, and sleeve 216 has an outer diameter D₂₁₆ that is less than diameter D₂₁₁. Housing 210 also includes a central throughbore or passage 215 that extends axially through base 211 and sleeve 216 between ends 210a, b. Passage 215 has a diameter D₂₁₅.

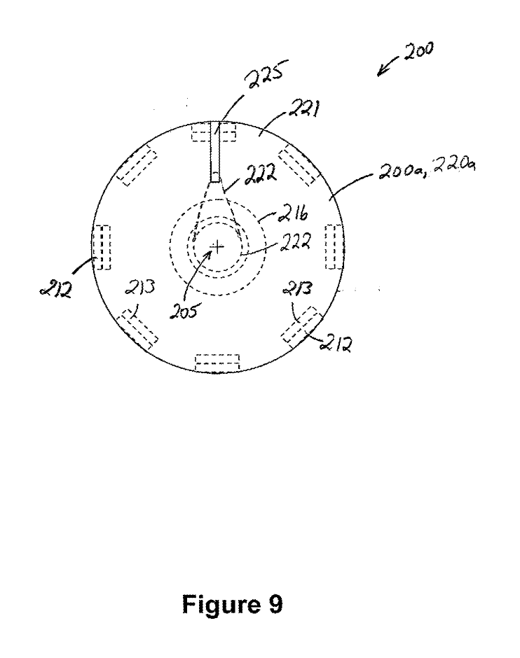

Base 211 has a proximal face 211a at end 210a, and a distal face 211b opposite face 211a and distal end 210a. As best shown in FIGS. 7 and 9, in this embodiment, a plurality of coupling arms or tabs 212 extend axially from distal face 211b towards end 210b. Arms 212 are uniformly, circumferentially spaced about the outer periphery of distal face 211b. Each arm 212 includes a fixed end 212a integral with base 211 at face 211b and a free end 212b distal face 211b. Free end 212b of each arm 212 includes an lip or ridge 213 extending radially inward from its corresponding arm 212. As will be described in more detail below, lips 213 releasably engage annular recess 103 of vascular sheath 100 previously described to restrict and/or prevent housing 210 from moving axially relative to vascular sheath 100 when sheath 100 is mounted to introducer 200. Arms 212 are made of a resilient material and act like springs, thereby allowing ends 212b to be flexed radially outward relative to fixed ends 212a to move lips 213 radially outward, and then allowed to relax and move back to their unflexed state shown in FIGS. 6 and 9. In this manner, lips 213 may be moved out of and into positive engagement with recess 103.

In general, housing 210 may be made of any suitable material(s) including, without limitation, metals (e.g., aluminum) and metal alloys (e.g., stainless steel, nitinol, etc.), polymers (e.g., plastics), composites (e.g., carbon fiber and epoxy matrix composite), or combinations thereof. However, housing 210 is preferably made of biocompatible, rigid materials, such as plastic, that allow housing 210 to be urged through a percutaneous access puncture and AV graft or fistula. In this embodiment, base 211 and sleeve 216 are monolithically formed, and thus, housing 210 is a single-piece or monolithic structure. However, in other embodiments, the introducer housing (e.g., housing 210) may be formed from multiple components that are coupled together (e.g., base 211 and sleeve 216 are separate components that are coupled together to form housing 210). In such embodiments, the component may comprise the same or different materials.

Referring still to FIGS. 6-9, stylet 220 extends along axis 205 between a first or proximal end 220a defining introducer end 200a and a second or distal end 220b defining introducer end 200b. In addition, stylet 220 includes an enlarged actuation head 221 at proximal end 220a, a curved, tapered tip 222 at distal end 220b, and an elongate shaft 223 extending between head 221 and tip 222. As best shown in FIG. 9, the proximal face 221a of head 221 includes a tip alignment indicator or marker 225 that is circumferentially aligned with tip 222. Thus, the operator of introducer 200 can utilize marker 225 to determine the orientation of curved tip 222 when curved tip 222 is not in the line of sight (e.g., tip 222 is disposed within the AV graft or fistula).

Shaft 223 extends through housing 210, however, head 211 and tip 223 are external housing 210-head 211 is axially proximal end 210a and base 211, and tip 223 is axially proximal end 210b. In this embodiment, shaft 223 is an elongate, solid, cylindrical rod. As best shown in FIG. 8, and as will be described in more detail below, axial force may be applied to head 211 by the operator of introducer 200 to move head 211, shaft 223, and tip 222 axially relative to housing 210.

In general, stylet 220 may be made of any suitable material(s) including, without limitation, metals (e.g., aluminum) and metal alloys (e.g., stainless steel, nitinol, etc.), polymers (e.g., plastics), composites (e.g., carbon fiber and epoxy matrix composite), or combinations thereof. However, stylet 220 is preferably made of biocompatible materials. Further, head 221 and shaft 223 preferably comprise rigid materials capable of transferring axial forces to tip 222 without substantial deformation. However, tip 222 preferably comprises a resilient, semi-rigid material that allows tip 222 to be flexed such that it can pass through vascular sheath 100 and the percutaneous access puncture and AV graft or fistula, but that also allows tip 222 to substantially maintain its curved shape within AV graft or fistula as it is manipulated therein. Examples of suitable materials for head 221 and shaft 223 include, without limitation, metals and metal alloys, composites, and polymers. Examples of suitable materials for tip 222 include, without limitation, polymers such as plastic and rubber materials. In this embodiment, head 221 and shaft 223 are monolithically formed, and tip 222 is connected to shaft 223 after shaft 223 is disposed in housing 210. However, in other embodiments, any two or more components of the stylet (e.g., stylet 220) may be formed from monolithic or coupled together. In such embodiments, the components coupled together may comprise the same or different materials.

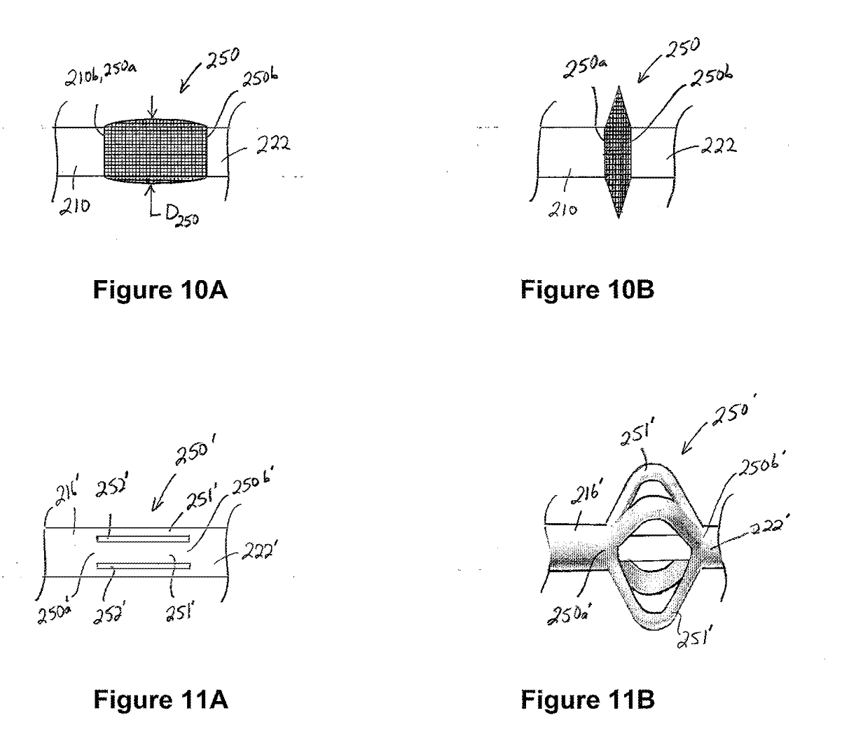

Referring now to FIGS. 6-8, radially expandable member 250 is disposed about shaft 223 and is axially positioned between housing distal end 210b and stylet tip 222. In particular, expandable member 250 has a proximal or first end 250a that axially abuts and is coupled to housing distal end 210b, and a distal or second end 250b that axially abuts and is coupled to tip 222. Expandable member 250 is controllably radially expanded and radially contracted by moving tip 222 axially relative to housing 210 via head 221 and shaft 223. In particular, member 250 expands radially outward from a first or unexpanded position shown in FIGS. 6, 7, and 10A to a second or radially expanded position shown in FIGS. 8 and 10B by moving tip 222 axially towards housing sleeve 216, thereby moving ends 250a, b axially together, and member 250 is radially contracted from the expanded position shown in FIGS. 8 and 10A to the unexpanded position shown in FIGS. 6, 7, and 9A by moving tip 222 axially away from housing 210, thereby moving ends 250a, b axially apart. Tip 222 is moved axially relative to housing sleeve 216 via actuation of head 221 relative to base 211. Specifically, to transition expandable member 250 from the unexpanded position to the expanded position, head 221 is pulled axially away from base 211 in the direction of arrow 230 to move tip 222 axially towards sleeve 216, and to transition expandable member 250 from the expanded position to the unexpanded position, head 221 is pushed axially toward base 211 in the direction of arrow 231 to move tip axially away from sleeve 216. As will be described in more detail below, in this embodiment, expandable member 250 is biased to the unexpanded position, and thus, has a tendency to urge head 221 and tip 222 axially away from base 211 and sleeve 216, respectively, towards the unexpanded position. In the unexpanded position, expandable member 250 preferably has an outer diameter D₂₅₀ that is the same or slightly greater than outer diameter D₂₁₆ of housing sleeve 216.

To enable such radial expansion and contraction, expandable member 250 preferably comprises a biocompatible, resilient, spring like material capable of being repeatedly transitioned between the expanded and unexpanded positions. For example, the expandable member (e.g., expandable member 250) may be made of a metal or metal alloy (e.g., nitinol), a polymer (e.g., plastic), or composite. In this embodiment, expandable member 250 is biased to the radially contracted and unexpanded position shown in FIGS. 6, 7, and 10A, and must be axially compressed into the radially expanded position shown in FIGS. 8 and 10B. In other words, expandable member 250 is relaxed in the radially contracted and unexpanded position, and unrelaxed and under axially compressive loads in the expanded position. Consequently, in this embodiment, expandable member 250 exerts no forces on housing 210 or tip 222 in the unexpanded position, but in the expanded position, exerts axial forces on housing 210 and tip 222 tending to urge them axially apart. In other embodiments, the expandable member (e.g., expandable member 250) may be oppositely biased (i.e., biased to the expanded position).

In general, any suitable radially expandable component or structure capable of being radially contracted and expanded as its ends are moved axially apart and together, respectively, may be employed for the expandable member (e.g., expandable member 250). For example, in the embodiment shown in FIGS. 6-10B, expandable member 250 is a wire mesh. As another example, in FIGS. 11A and 11B, expandable member 250′ comprises a plurality elongate axial strips 251′ defined by a plurality of axially spaced slits 252′ in the introducer sleeve 216′. As ends 250a′, b′ of expandable member 250′ are move axially together, strips 251′ are axially compressed and buckle, thereby expanding radially outward. In the embodiment shown in FIGS. 11A and 11B, strips 251′ are integral with sleeve 216′ and tip 222′. Other examples of devices suitable for use as the expandable member (e.g., expandable member 250) include, without limitation, wire baskets, wire screens, etc. To ensure the expandable member (e.g., expandable member 250, 205′) expands radially outward when it is axially compressed, the expandable member may be convex or outwardly bowed between its ends (e.g., ends 250a, b, 250a′, b′) as shown in FIG. 10A such that it is predisposed to buckle radially outward and expand radially outward.

As best shown in FIG. 7, in this embodiment, introducer 200 includes a locking mechanism 260 that controllably and releasably couples shaft 223 and housing 210. Specifically, locking mechanism 260 has a locked position in which head 221, shaft 223, and tip 222 are restricted from moving axially relative to housing 210, and an unlocked position in which head 221, shaft 223, and tip 222 may move axially relative to housing 210. As previously described, in this embodiment, expandable member 250 is biased to the unexpanded position shown in FIGS. 6, 7, and 10A. Thus, when expandable member 250 is in the radially expanded position, it has a tendency to return to its relaxed, unexpanded position. Consequently, in this embodiment, locking mechanism 260 is used to releasably lock shaft 223 and tip 222 relative to housing 210, thereby preventing relative axial movement therebetween, when expandable member 250 is in the radially expanded position shown in FIG. 8. For example, head 221 is urged axially away from base 211 to transition expandable member 250 from the unexpanded position to expanded position. As expandable member 250 is axially compressed and transitioned to the expanded position, it seeks to return to the unexpanded position. However, locking mechanism 260 locks the position of head 221, tip 222, and shaft 223 relative to housing 210, thereby resisting the axial forces exerted by expandable member 250 on sleeve 216 and tip 222, and maintaining expandable member 250 in the expanded position, even if axial forces urging head 221 away from base 211 cease.

Once locking mechanism 260 is locked, application of a subsequent axial force on head 211 will transition locking mechanism 260 to an unlocked position in which head 211, tip 222, and shaft 223 are allowed to move axially relative to housing 210 under the axial forces exerted by the operator on head 221 and/or the axial forces exerted by the compressed expandable member 250. In other words, once locking mechanism 260 unlocks, expandable member 250 is allowed to return to the relaxed, unexpanded position thereby moving tip 222 axially away from sleeve 216. In general, locking mechanism 260 may comprise any suitable mechanism for releasably locking the axial position of shaft 223 and tip 222 relative to housing 210. Examples of suitable locking mechanisms are disclosed in U.S. patent application Ser. No. 11/302,951, which is hereby incorporated herein by reference in its entirety. Another example of a suitable locking mechanism is the releasably locking mechanism utilized in ball point pens to lock the writing tip of the pen in the extended position relative to the pen housing, and then allow the writing tip to retract into the pen housing by application of a subsequent axial force. In other embodiments where the expandable member (e.g., expandable member 250) is biased to the expanded position, the locking mechanism (e.g., locking mechanism 260) is configured to releasably lock the shaft (e.g., shaft 223) and the tip (e.g., tip 222) relative to the housing (e.g., housing 210) when the expandable member is in the unexpanded position.

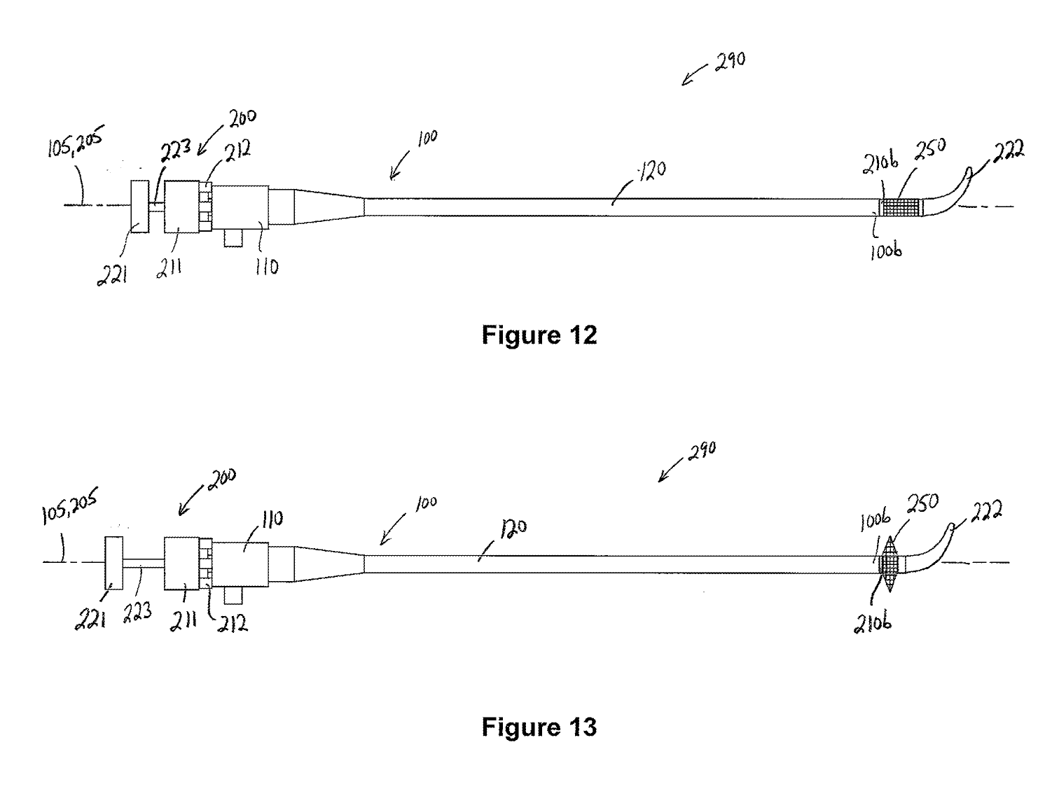

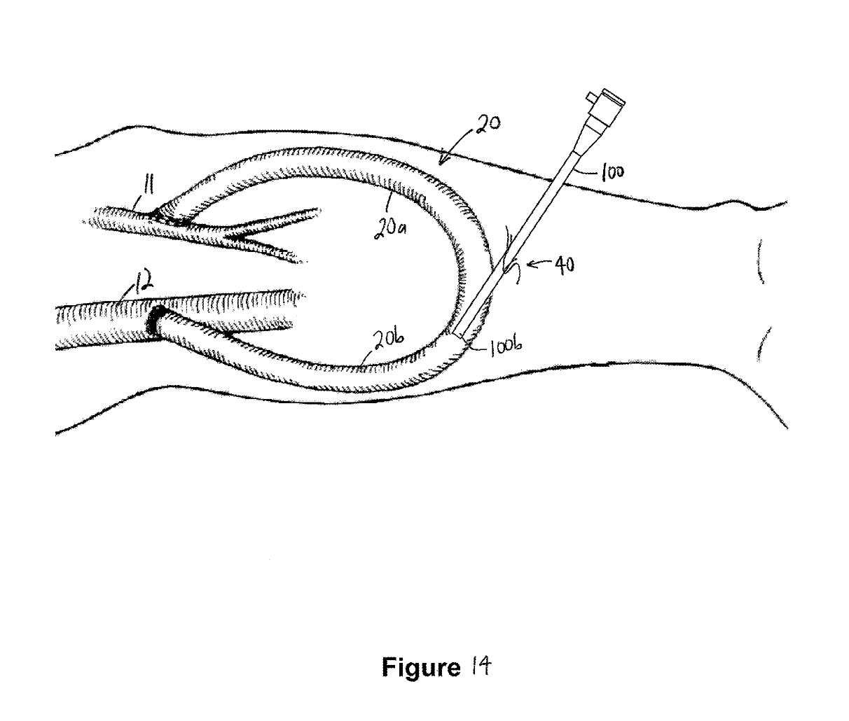

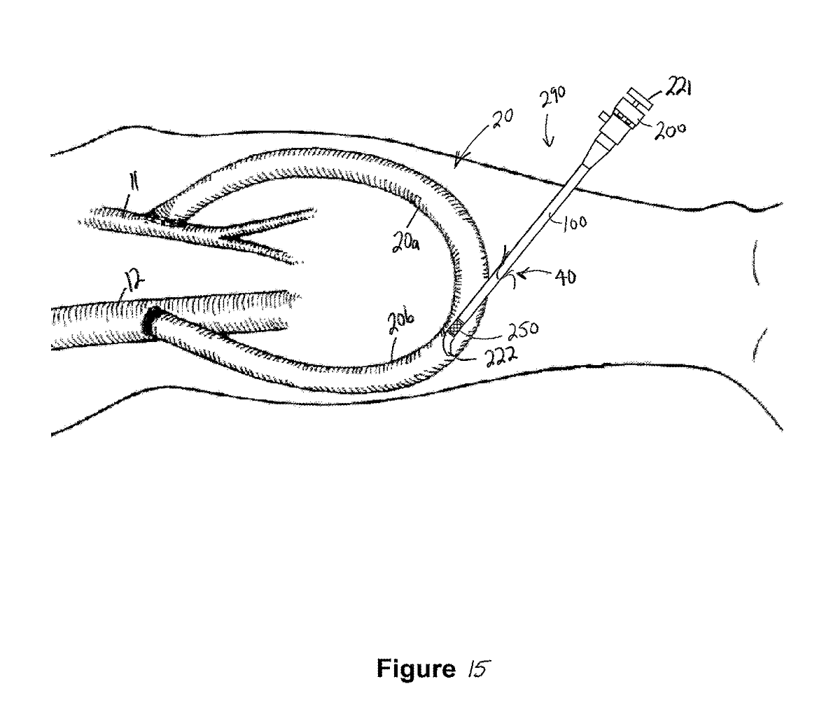

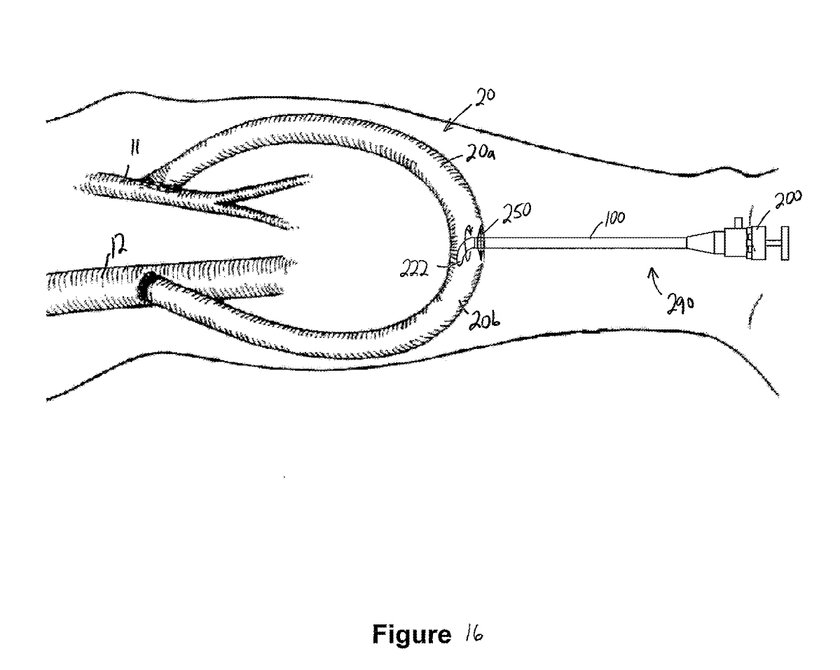

Referring now to FIGS. 12 and 13, vascular sheath 100 previously described is shown coaxially mounted to introducer 200 to form a vascular sheath-introducer assembly 290. To position introducer 200 within vascular sheath 100, tip 222 may be axially inserted into sheath throughbore 101 at end 100a, and axially advanced through throughbore 101 until it emerges from throughbore 101 at end 100b and body 211 engages sheath end 100a. Introducer 200 and sheath 100 are sized and configured such that sleeve 216, tip 222, and expandable member 250 pass completely through sheath 100, and body 211 axially abuts sheath end 100a simultaneous with positive engagement of lips 213 and annular recess 103. In particular, sleeve outer diameter D₂₁₆ is less than sheath inner diameter D₁₀₁. In addition, expandable member 250 is configured such that it may be axially advanced through sheath 100 in its unexpanded position. For example, diameter D₂₅₀ of expandable member 250 in its unexpanded position may be less than diameter D₁₀₁, or if diameter D₂₅₀ of expandable member 250 in its unexpanded position is slightly greater than diameter D₁₀₁, expandable member 250 may be radially compressed as it slidingly engages the inner surface of sheath 100. To advance curved tip 222 through sheath 100, tip 222 may flex and be urged radially inward by the inner surface of sheath 100 disposed at diameter D₁₀₁. As end 100a moves axially into contact with distal ends 212b of arms 212, ends 212b are urged radially outward relative to ends 212a to allow end 100a to pass between arms 212. As end 100a passes between arms 212, lips 213 slidingly engage the outer surface of body 110 and then snap radially inward when aligned with recess 103, thereby releasably locking sheath 100 to introducer 200. With sheath 100 mounted to introducer 200, introducer 200 may be used to manipulate the position and angular orientation of sheath 100.

Sleeve length L₂₁₆ is greater than sheath length L₁₀₀, and thus, expandable member 250 and tip 222 extend axially from sheath 100. Consequently, sheath 100 does not restrict or obstruct the transition of expandable member between the radially expanded and unexpanded positions. Thus, expandable member 250 may be transitioned between the unexpanded position shown in FIG. 12 and the expanded position shown in FIG. 13 while introducer 200 is disposed within vascular sheath 100.

As shown in FIGS. 14-20, vascular sheath 100 and introducer 200 enable controlled percutaneous access to both the venous limb and the arterial limb of an AV graft or fistula with a single puncture, while simultaneously offering the potential to reduce the likelihood of inadvertent loss of access to the AV graft or fistual. Referring first to FIG. 14, vascular sheath 100 is shown extending through a single puncture 40 into AV graft 20. In particular, vascular sheath 100 is positioned to provide access to the venous limb 20b of AV graft 20. Namely, proximal end 100a is external the patient and distal end 100b is disposed in venous limb 20b. Moving now to FIG. 15, while the position of sheath 100 in venous limb 20b is maintained, introducer 200, with expandable member 250 in the unexpanded position, is axially inserted and advanced through vascular sheath 100 until tip 222 and expandable member 250 emerge from sheath distal end 100b into venous limb 20b, which occurs simultaneous with axial engagement of introducer base 211 and sheath proximal end 100a and coupling of sheath 100 and introducer 200 via mating engagement of arms 212 and annular recess 103.

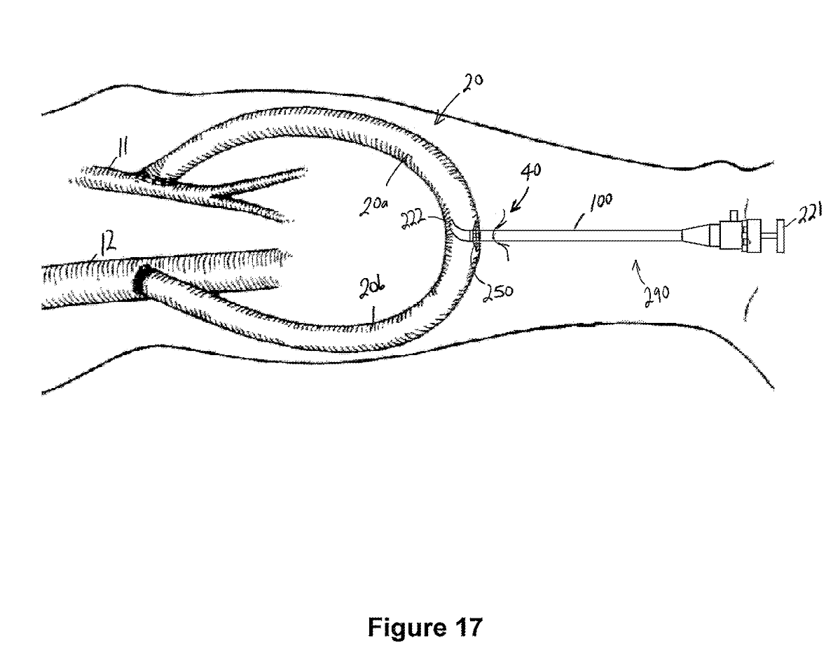

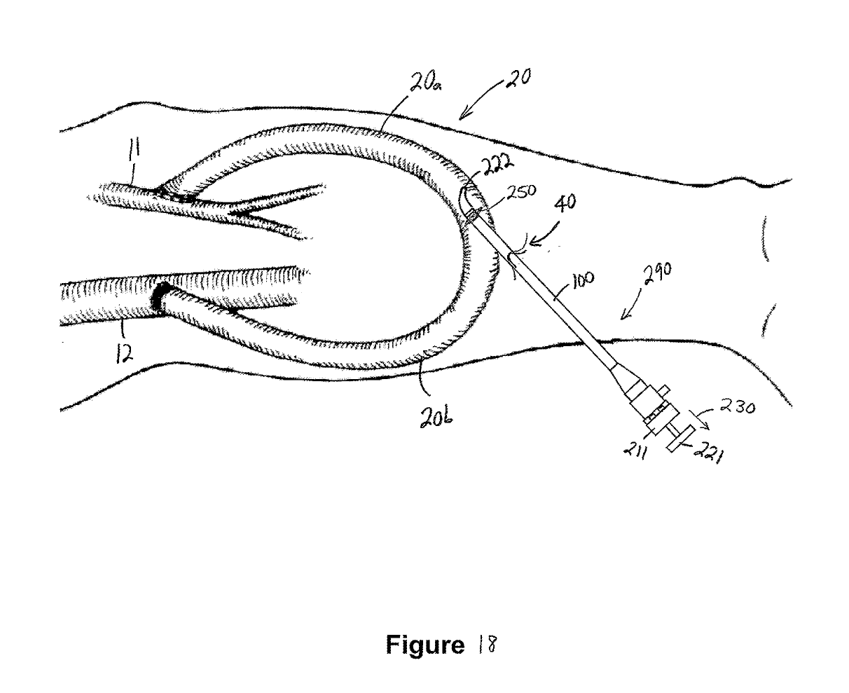

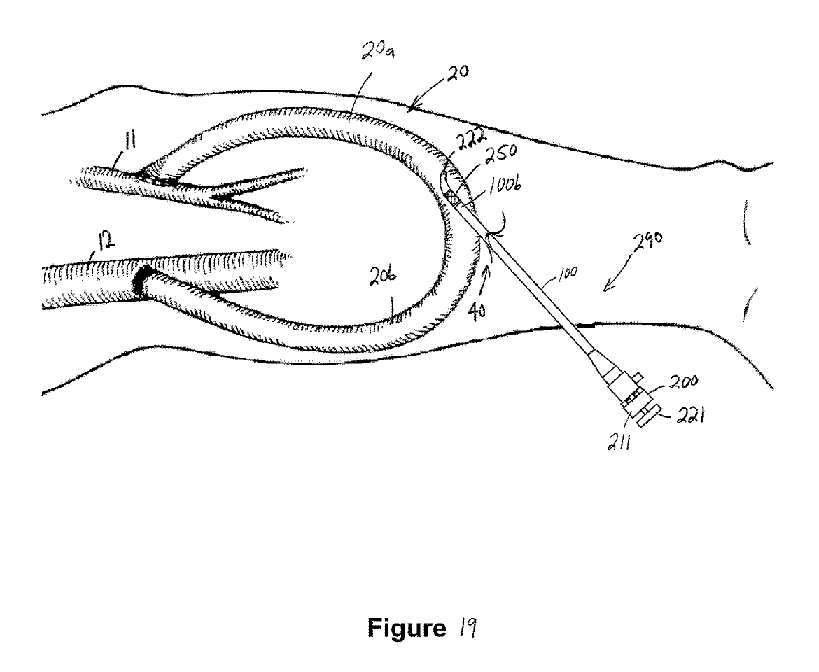

As shown in FIGS. 16 and 17, expandable member 250 is transitioned to the expanded position and sheath-introducer assembly 290 is pulled back through puncture 40. However, tip 222 and expandable member 250 are restricted and/or prevented from exiting AV graft 20 by the radially expanded member 250. Specifically, in the radially expanded position, expandable member 250 bears against the inner wall of AV graft 20, thereby restricting and/or preventing expandable member 250 and tip 222 from inadvertently exiting AV graft 20 and puncture 40. Engagement of expandable member 250 and the inner wall of AV graft 20 is detected by the operator as an increase in resistance to the retraction of sheath-introducer assembly 290 from puncture 40. Next, while maintaining expandable member 250 in the expanded position (e.g., with locking mechanism 260), tip 222 is rotated about axes 105, 205 such that tip 222 is pointed towards the arterial limb 20a as shown in FIG. 18. Marker 225 on head 221 may be used by the operator to determine the orientation of tip 222. In general, tip 222 may be rotated by rotating introducer 200 with head 221 or rotating the entire sheath-introducer assembly 290.

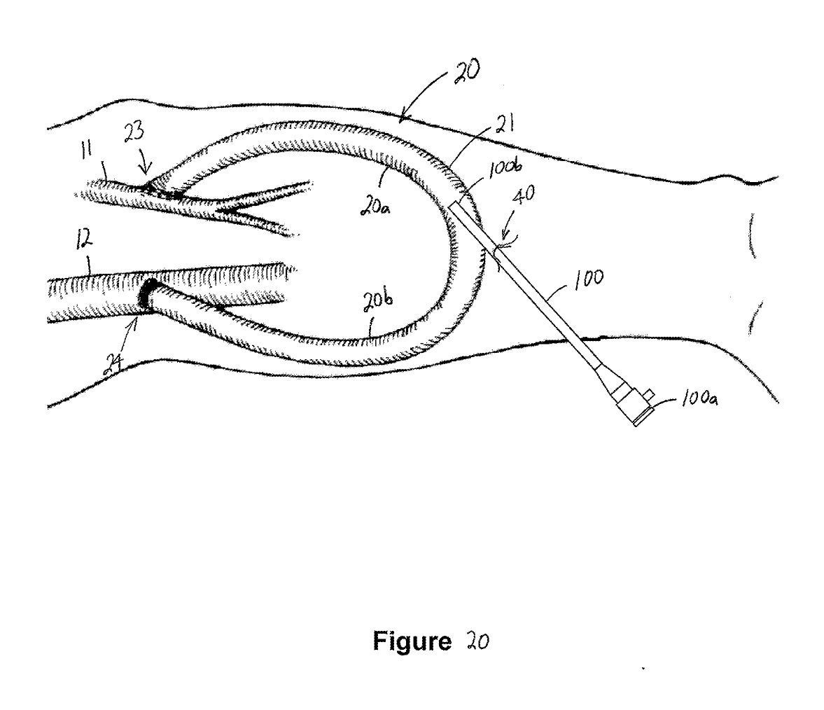

Moving now to FIG. 19, with tip 222 pointing into and generally aligned with arterial limb 20a, sheath-introducer assembly 290 is pushed through puncture 40 and AV graft 20 and expandable member 250 is transitioned to the unexpanded position. Accordingly, tip 222 and sheath distal end 100b are urged into arterial limb 20a. By aligning tip 222 with arterial limb 20a prior to urging sheath-introducer assembly 290 further into AV graft 20, tip 222 provides a guide to ensure sheath-introducer assembly 290 advances in the desired direction. With sheath distal end 100b sufficiently positioned to provide access to arterial limb 20a, sheath 100 is maintained in position as introducer 200 is decoupled and removed from sheath 100 as shown in FIG. 20. In the manner described, introducer 200 is used to percutaneously adjust the position of vascular sheath 100, through a single puncture 40, between one position providing access to venous limb 20b and another position providing access to arterial limb 20a. It should be appreciated, that the same basic procedure may also be used to reposition vascular sheath 100 from a position providing access to arterial limb 20a to a position providing access to venous limb 20b through single puncture 40.

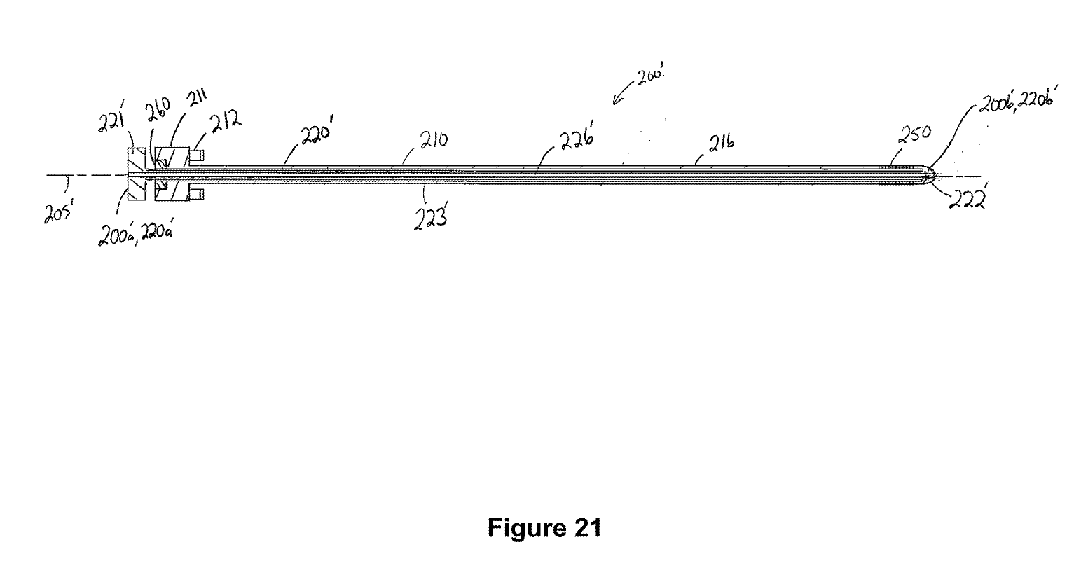

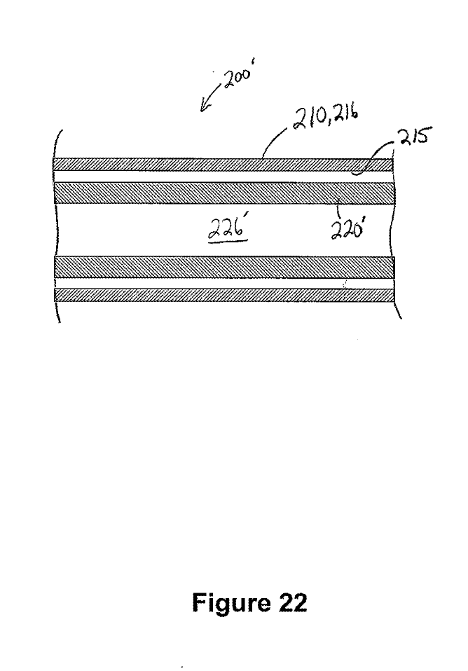

In the embodiment of introducer 200 previously described and shown in FIGS. 6-8, stylet 220 is a solid structure between ends 220a, 220b, and thus, stylet 220 is not adapted to accept a guide wire for initial positioning and/or repositioning of vascular sheath 100 within the AV graft 20 through puncture 40. Thus, sheath 100 may be initially positioned in the AV graft 20 as shown in FIG. 14 using a conventional introducer that is passed into the patient over a guide wire, and then introducer 200 may be employed to reposition sheath 100 between the venous and arterial limbs 20b, a. However, referring now to FIGS. 21 and 22, another embodiment of an introducer 200′ suitable for use with a guide wire for the initial placement and/or repositioning of a vascular sheath (e.g., sheath 100) within an AV graft or fistula (e.g., AV graft 20) is shown. Introducer 200′ is substantially the same as introducer 200 previously described with two exceptions—stylet 220′ of introducer 200′ includes an elongate throughbore 226′ extending axially between ends 220a′, 220b′, and stylet 220′ does not include a tapered curved tip (e.g., tip 222). Specifically, introducer 200′ has a central or longitudinal axis 205′, a first or proximal end 200a′, and a second or distal end 200b′. In addition, introducer 200′ includes a radially outer housing 210 as previously described, a radially inner stylet 220′ moveably disposed in housing 210, and expandable member 250 as previously described disposed about stylet 220′ proximal end 200b′. Further, stylet 220′ has a first or proximal end 220a′, a second or distal end 220b′, and includes an enlarged actuation head 221′ at proximal end 220a′, smooth, rounded tip 222′ at distal end 220b′, and an elongate, tubular shaft 223′ extending between head 221′ and tip 222′. Throughbore 226′ extends through head 221′, shaft 223′, and tip 222′ between ends 220a′, 220b′. Expandable member 250 is disposed about shaft 223′ between housing distal end 210b and stylet tip 222′ and functions as previously described. A locking mechanism 260 as previously described controllably and releasably couples shaft 223′ and housing 210.

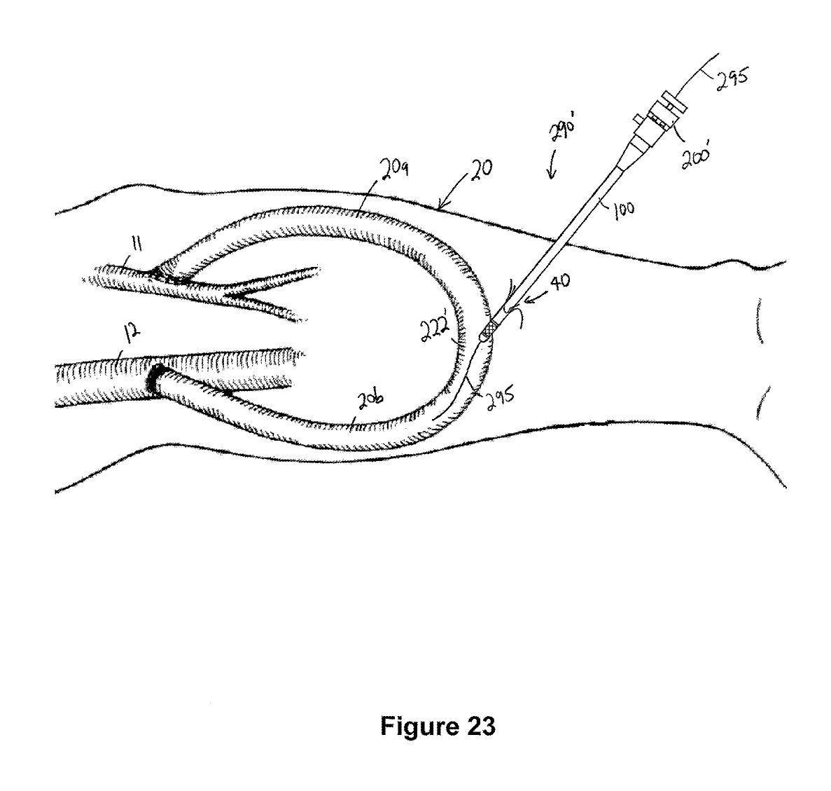

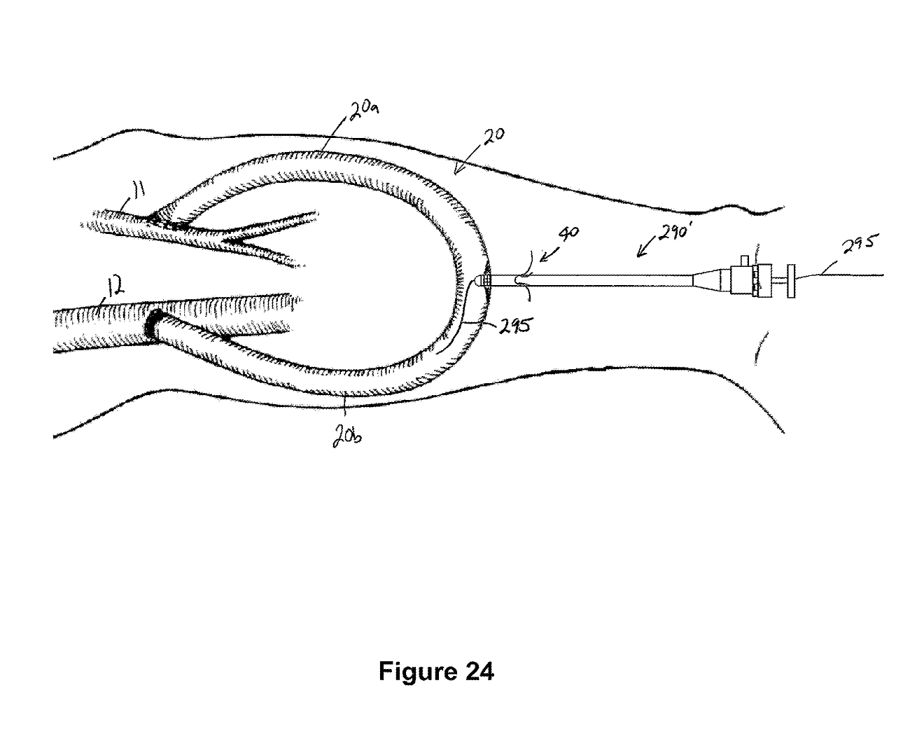

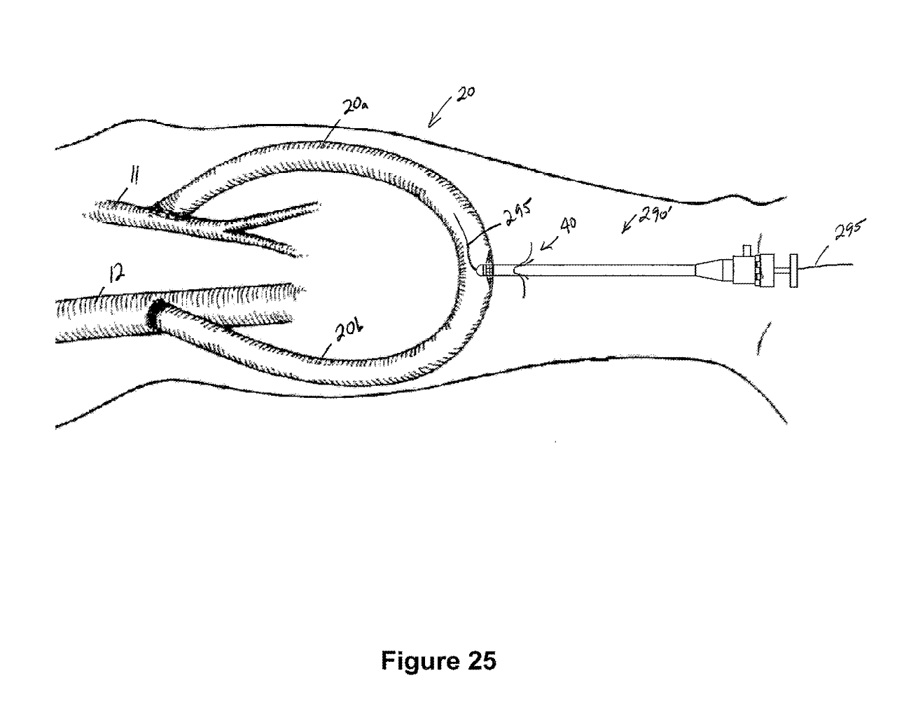

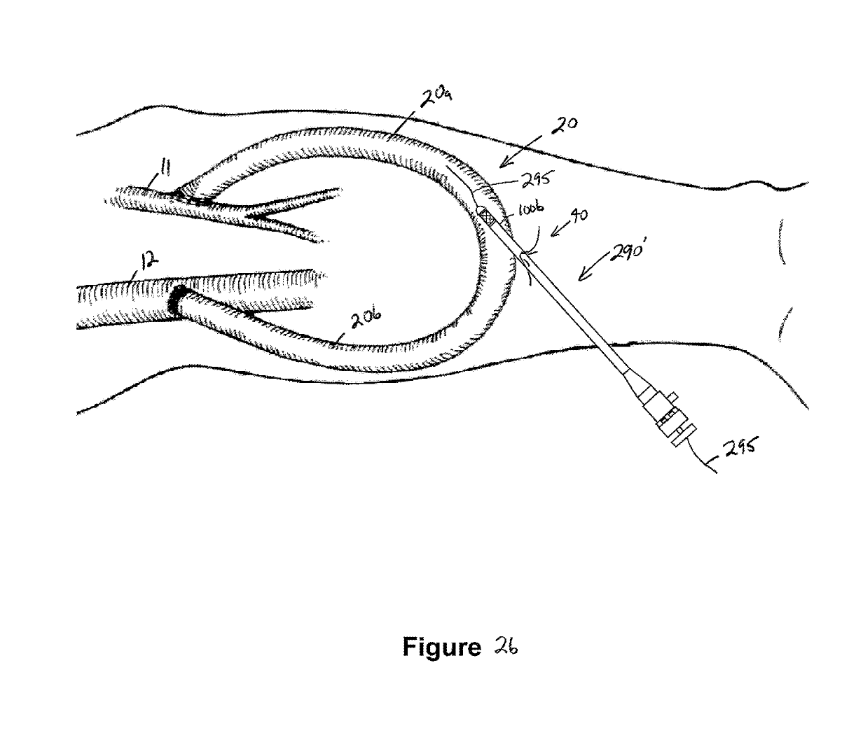

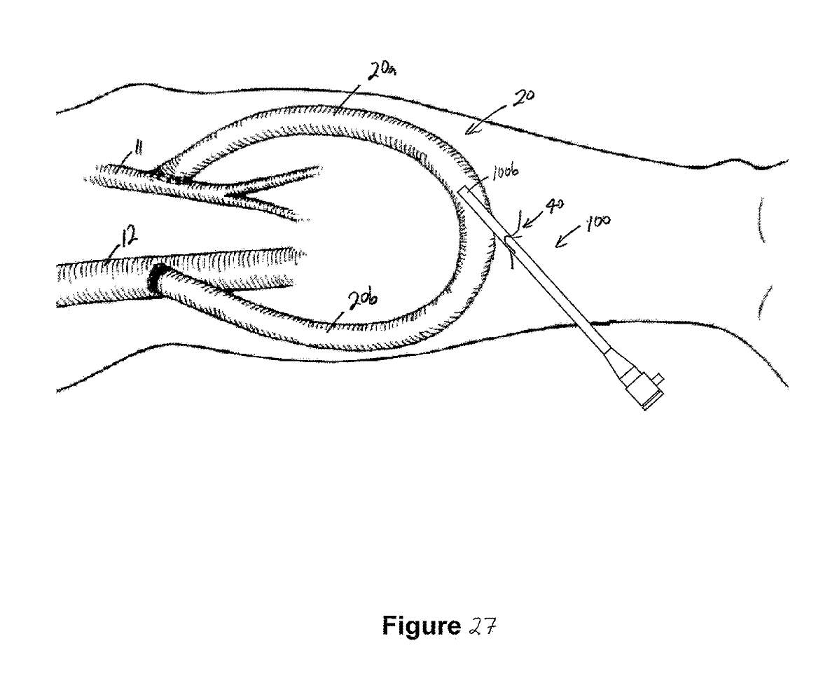

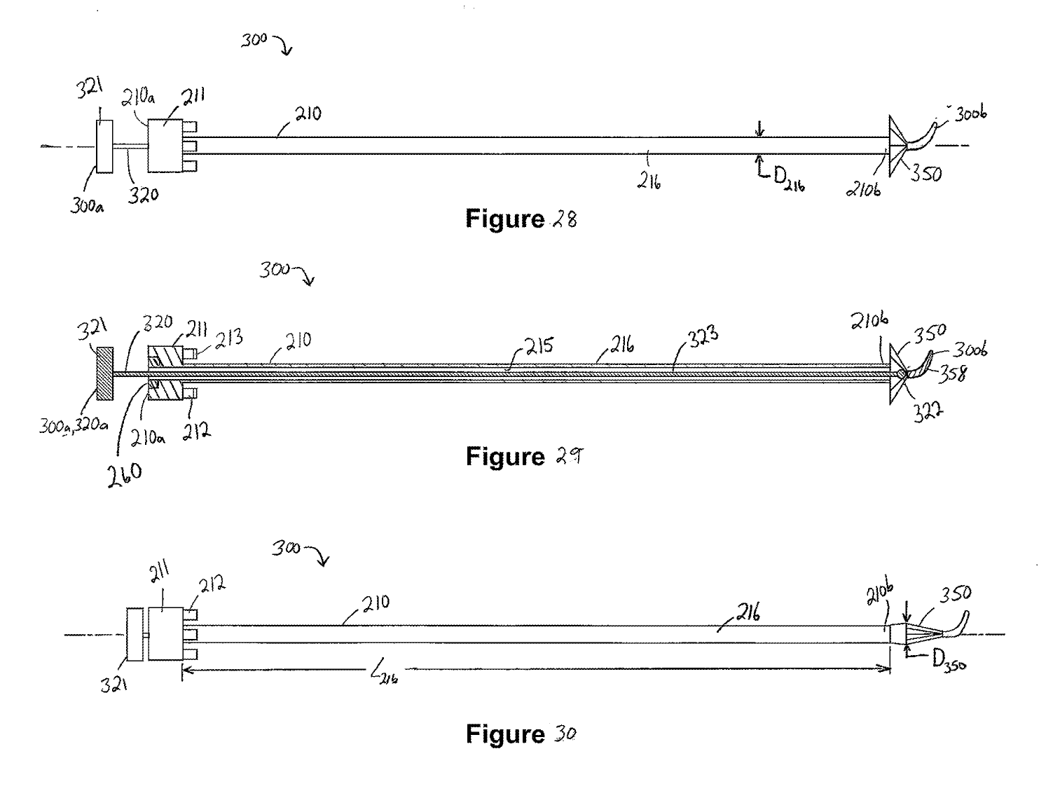

Introducer 200′ is designed for use with a guide wire 295. For example, as shown in FIG. 23, a sheath-introducer assembly 290′ including sheath 100 previously described mounted to introducer 200′, may be passed over a guide wire 295 extending into AV graft 20 to initially position the vascular sheath 100 within the AV graft 20. Further, guide wire 295 may also be employed to reposition vascular sheath 100 from one limb 20a, b to the other limb 20a, b. In particular, with sheath 100 and guide wire 295 in the venous limb 20b of the AV graft 20, introducer 200′ may be advanced over guide wire 295 and loaded coaxially into sheath 100. Then, as shown in FIG. 24, expandable member 250 may then be transitioned to the expanded position, and then, leaving guide wire 295 in place (extending into the venous limb 20b), sheath-introducer assembly 290′ is pulled back through puncture 40. However, expandable member 250 restricts and/or prevents assembly 290′ from exiting AV graft 20. Engagement of expandable member 250 and the inner wall of AV graft 20 is detected by the operator as an increase in resistance to the retraction of sheath-introducer assembly 290′. Next, as shown in FIG. 25, while maintaining expandable member 250 in the expanded position, guide wire 295 is carefully pulled back under fluoroscopic guidance until its distal tip reaches introducer distal end 200b, and then guide wire 295 is manipulated towards the arterial limb 20a of the AV graft 20. Moving now to FIG. 26, guide wire 295 is extended into the arterial limb 20a, expandable member 250 is transitioned to the unexpanded position, and assembly 290′ is advanced over wire 295 into the arterial limb 20a. With sheath distal end 100b sufficiently positioned to provide access to arterial limb 20a, sheath 100 is maintained in position as introducer 200′ is decoupled and removed from sheath 100 as shown in FIG. 27.

Although the option of a hollow stylet (e.g., stylet 220′ with throughbore 226) for use in conjunction with a guide wire is included in select embodiments described herein. In general, a hollow stylet may be employed in any introducer embodiment described herein to enable “over-the-wire” initial positioning and/or repositioning of the vascular sheath (e.g., sheath 100) within the AV graft or fistula (e.g., AV graft 20).

Referring now to FIGS. 28-30, an embodiment of a vascular sheath introducer 300 is shown. In general, introducer 300 is a device used to position a vascular sheath, such as vascular sheath 100 previously described, into a vessel, AV graft, or AV fistula, and manipulate the vascular sheath within the vessel, AV graft, or AV fistula. As will be described in more detail below, introducer 300 enables a vascular sheath (e.g., vascular sheath 100) to be pivoted or swiveled back and forth within the lumen of an AV graft or fistula between the venous limb and arterial limb as many times as necessary, while reducing likelihood of inadvertent access loss and associated problems. As a result, embodiments of introducer 300 allow the operator to access both the arterial and venous limb of an AV graft or fistula via a single puncture. Further, embodiments of introducer 700 allow the operator to access an AV graft or AV fistula at any level and work in antegrade and retrograde fashions, via a single puncture.

In this embodiment, introducer 300 has a central or longitudinal axis 305, a first or proximal end 300a, and a second or distal end 300b. In addition, introducer 300 includes a radially outer housing 210 as previously described, a radially inner stylet 320 extending through housing 210, and a distal, radially expandable member 350 disposed about stylet 320 proximal end 300b. Housing 210, stylet 320, and expandable member 350 are each coaxially aligned with longitudinal axis 305. Further, stylet 320 is adapted to move axially relative to housing 210.

Referring still to FIGS. 28-30, stylet 320 extends along axis 305 between a first or proximal end 320a defining introducer end 300a and a second or distal end 320b disposed within expandable member 350. In addition, stylet 320 includes an enlarged actuation head 321 at proximal end 320a, an engagement member 322 at distal end 320b, and an elongate member 323 extending between head 321 and engagement member 322. Elongate member 323 extends through housing 210, however, head 321 and engagement member 322 are external housing 210-head 321 extends axially from end 210a and base 211, and engagement member 322 extends axially from end 210b. As best shown in FIG. 29, engagement member 322 axially abuts the inner surface of expandable member 350. As will be described in more detail below, axial force may be applied to head 321 by the operator of introducer 300 to move head 321, elongate member 323, and engagement member 322 axially relative to housing 210. Thus, elongate member 323 functions to transmit axial forces between head 321 and engagement member 322. Engagement member 322 bears against the inner surface of expandable member 350 and transfers axial forces to expandable member 350. To reduce the likelihood of tearing or otherwise damaging the flexible expandable member 350 during application of axial forces to expandable member 350, engagement member 323 is preferably configured to transfer axial forces to expandable member 350 through a relatively smooth, large contact surface area. Thus, in this embodiment, engagement member 323 is a smooth surface, spherical ball. Further, in this embodiment, elongate member 323 is a semi-rigid wire capable of transmitting axial forces from head 321 to engagement member 322. However, in other embodiments, elongate member 323 may be a rigid shaft or hollow tubular.

In general, stylet 320 may be made of any suitable material(s) including, without limitation, metals (e.g., aluminum) and metal alloys (e.g., stainless steel, nitinol, etc.), polymers (e.g., plastics), composites (e.g., carbon fiber and epoxy matrix composite), or combinations thereof. However, stylet 320 is preferably made of biocompatible materials. Further, head 321, elongate member 323, and engagement member 322 preferably comprise rigid or semi-rigid materials capable of transferring axial forces to expandable member 350 without substantial deformation. Examples of suitable materials for head 321, elongate member 323, and engagement member 322 include, without limitation, metals and metal alloys, composites, and polymers. However, in other embodiments, any two or more components of the stylet (e.g., stylet 220) may be formed from monolithic or coupled together. In such embodiments, the components coupled together may comprise the same or different materials.

Referring now to FIGS. 31A and 31B, radially expandable member 350 is coupled to housing distal end 210b. In particular, expandable member 350 has a proximal or first end 350a that is fixedly attached to housing distal end 210b, and a distal or second end 350b. A curved, tapered tip 358 extends from expandable member end 350b to introducer distal end 300b. In this embodiment, tip 358 is integral and monolithically formed with expandable member 350. As best shown in FIG. 28, tip alignment indicator or marker 225 on head 321 is circumferentially aligned with tip 358. Thus, the operator of introducer 300 can utilize marker 225 to determine the orientation of curved tip 358 when curved tip 358 is not in the line of sight (e.g., tip 358 is disposed within the AV graft or fistula).

Referring still to FIGS. 31A and 31B, in this embodiment, expandable member 350 is a generally hollow, conical structure including an annular base or flange 351 extending radially outward from end 350a and a frustoconical portion 352 extending from end 350b to flange 351. Flange 351 includes an opening 353 at end 350a that is concentric with housing throughbore 215. Elongate member 320 extends through opening 353 into the interior of expandable member 350.

In this embodiment, expandable member 350 is controllably radially contracted by moving engagement member 322 axially into engagement with the inner surface of frustoconical portion 352 and applying axial force to frustoconical portion 352 with engagement member 322 to push end 350b axially away from end 350a as shown in FIG. 31B. Further, expandable member 350 is controllably radially expanded by axially retracting engagement member 322 and allowing end 350b to move axially towards end 350a as shown in FIG. 31A. It should be appreciated that engagement member 322 is moved axially via head 321 and elongate member 323. As will be described in more detail below, in this embodiment, expandable member 350 is biased to the radially expanded position. Thus, when axial forces are removed form head 321, the axial spring forces generated by expandable member 350 in the unexpanded position may be sufficient to move engagement member 322, elongate member 323, and head 321 axially to allow expandable member 350 to transition to the expanded position. In the unexpanded position, expandable member 350 preferably has an outer diameter D₃₅₀ that is the same or slightly greater than outer diameter D₂₁₆ of housing sleeve 216.

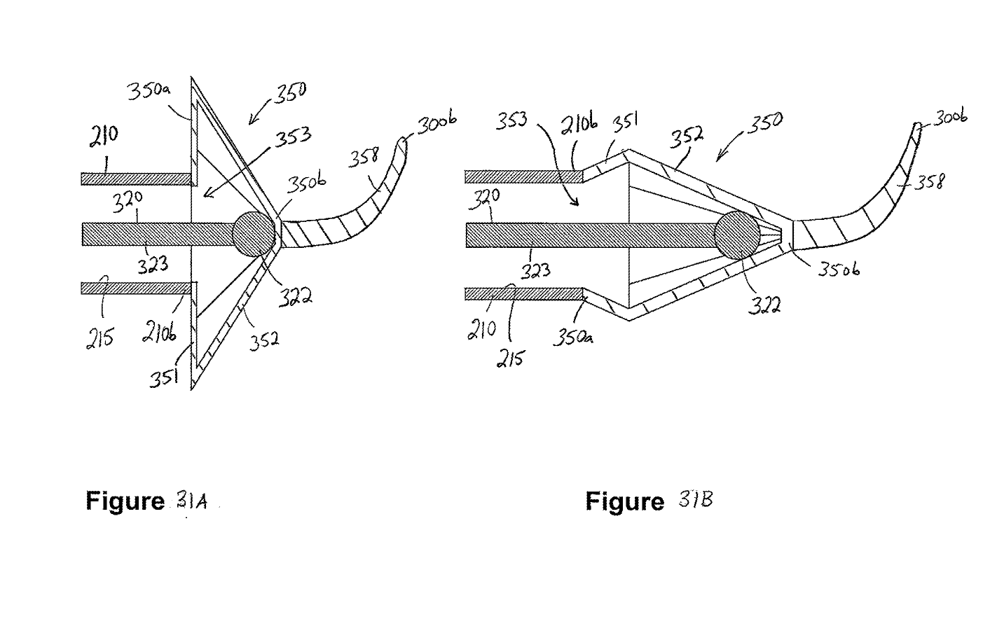

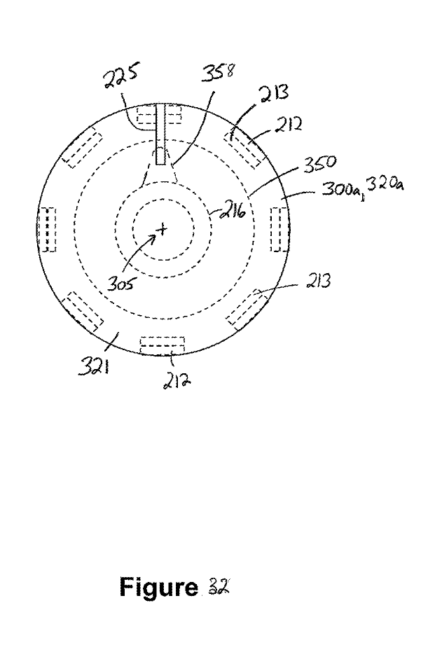

To enable such radial expansion and contraction, expandable member 350 preferably comprises a biocompatible, resilient, flexible material capable of being repeatedly stretched and transitioned between the expanded and unexpanded positions. For example, the expandable member (e.g., expandable member 350) may be made of a flexible polymer or rubber material. Tip 358 preferably comprises similar materials. In this embodiment, expandable member 350 is biased to the radially expanded position shown in FIGS. 28 and 31A, and must be axially stretched into the unexpanded position shown in FIGS. 30 and 31B. In other words, expandable member 350 is relaxed in the radially expanded position, and unrelaxed and under axially tensile loads in the unexpanded position. Consequently, in this embodiment, expandable member 350 exerts no forces on engagement member 322 in the expanded position, but in the expanded position, exerts axial forces on engagement member 222 tending to urge it axially towards housing end 210a. To aid in managing the axial biasing forces generated by expandable member 350 in the unexpanded position, a locking mechanism, such as locking mechanism 260 previously described, may be employed to releasably lock expandable member 350 in the unexpanded position. In other embodiments, the expandable member (e.g., expandable member 350) may be oppositely biased (i.e., biased to the expanded position). In such embodiments, the locking mechanism (e.g., locking mechanism 260) is configured to releasably lock the expandable member in the expanded position.

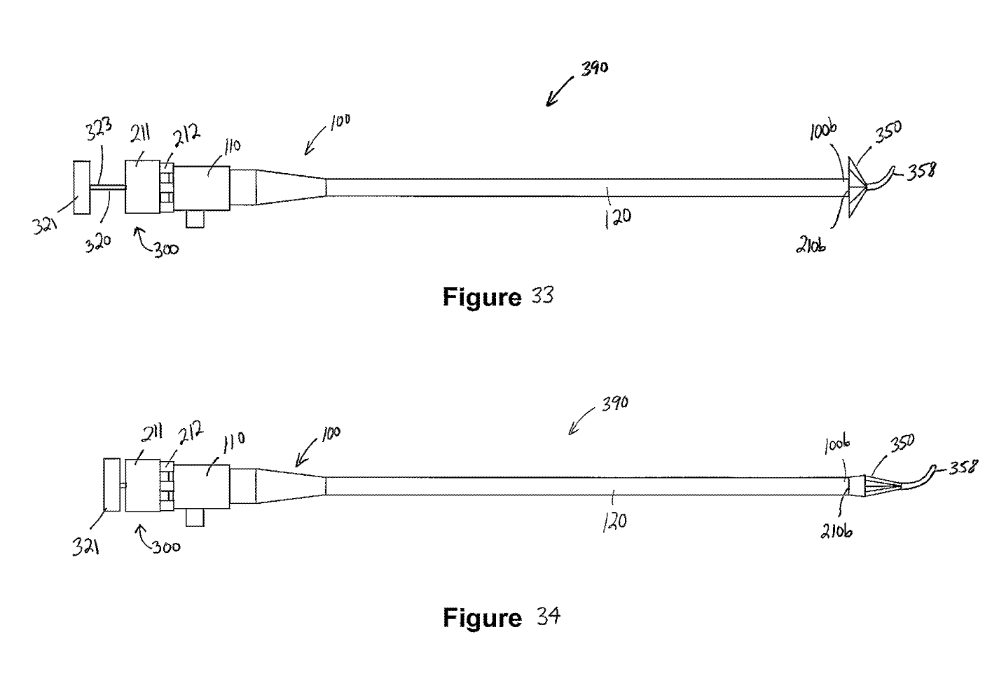

Referring now to FIGS. 33 and 34, vascular sheath 100 previously described is shown coaxially mounted to introducer 300 to form a vascular sheath-introducer assembly 390. To position introducer 300 within vascular sheath 100, expandable member 350 is transitioned to the unexpanded position shown in FIGS. 30 and 31B and tip 358 is axially inserted into sheath throughbore 101 at end 100a, and axially advanced through throughbore 101 until it emerges from throughbore 101 at end 100b and body 211 engages sheath end 100a. Introducer 300 and sheath 100 are sized and configured such that sleeve 216, tip 358, and expandable member 350 pass completely through sheath 100, and body 211 axially abuts sheath end 100a simultaneous with positive engagement of lips 213 and annular recess 103. In particular, sleeve outer diameter D₂₁₆ is less than sheath inner diameter D₁₀₁. In addition, expandable member 350 is configured such that it may be axially advanced through sheath 100 in its unexpanded position. For example, diameter D₃₅₀ of expandable member 350 in its unexpanded position may be less than diameter D₁₀₁, or if diameter D₃₅₀ of expandable member 350 in its unexpanded position is slightly greater than diameter D₁₀₁, expandable member 350 may be radially compressed as it slidingly engages the inner surface of sheath 100. To advance curved tip 358 through sheath 100, tip 358 may flex and be urged radially inward by the inner surface of sheath 100 disposed at diameter D₁₀₁. As end 100a moves axially into contact with distal ends 212b of arms 212, ends 212b are urged radially outward relative to ends 212a to allow end 100a to pass between arms 212. As end 100a passes between arms 212, lips 213 slidingly engage the outer surface of body 110 and then snap radially inward when aligned with recess 103, thereby releasably locking sheath 100 to introducer 300. With sheath 100 mounted to introducer 300, introducer 300 may be used to manipulate the position and angular orientation of sheath 100.

In this embodiment, sleeve length L₂₁₆ measured from head 211 to end 210b is the same as sheath length L₁₀₀, and thus, expandable member 350 and tip 358 extend axially from sheath 100. Consequently, sheath 100 does not restrict or obstruct the transition of expandable member between the radially expanded and unexpanded positions. Thus, expandable member 350 may be transitioned between the unexpanded position while introducer 300 is disposed within vascular sheath 100. As shown in FIG. 33, since sleeve length L₂₁₆ is the same as sheath length L₁₀₀, expandable member end 350a is axially adjacent sheath distal end 100b.

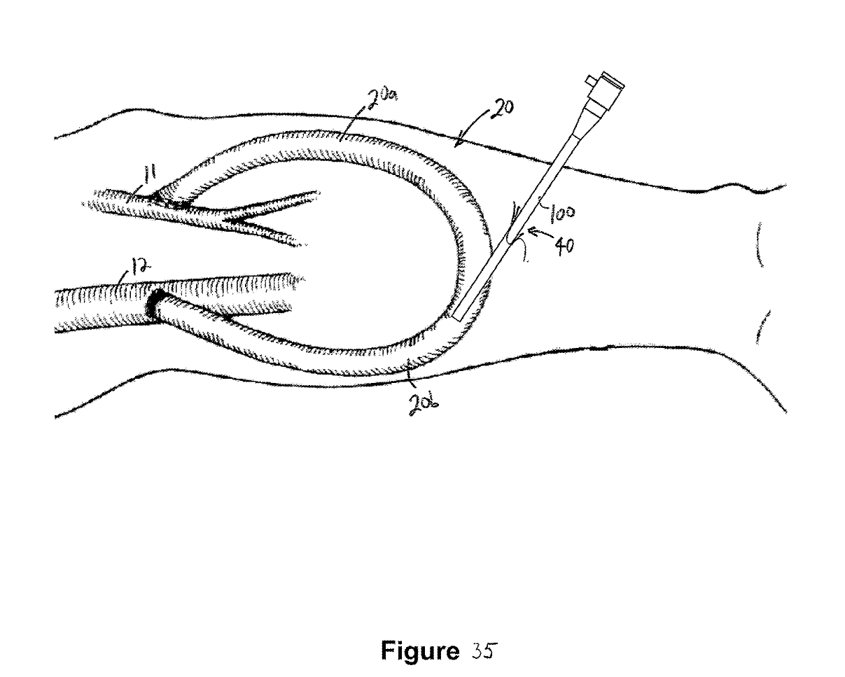

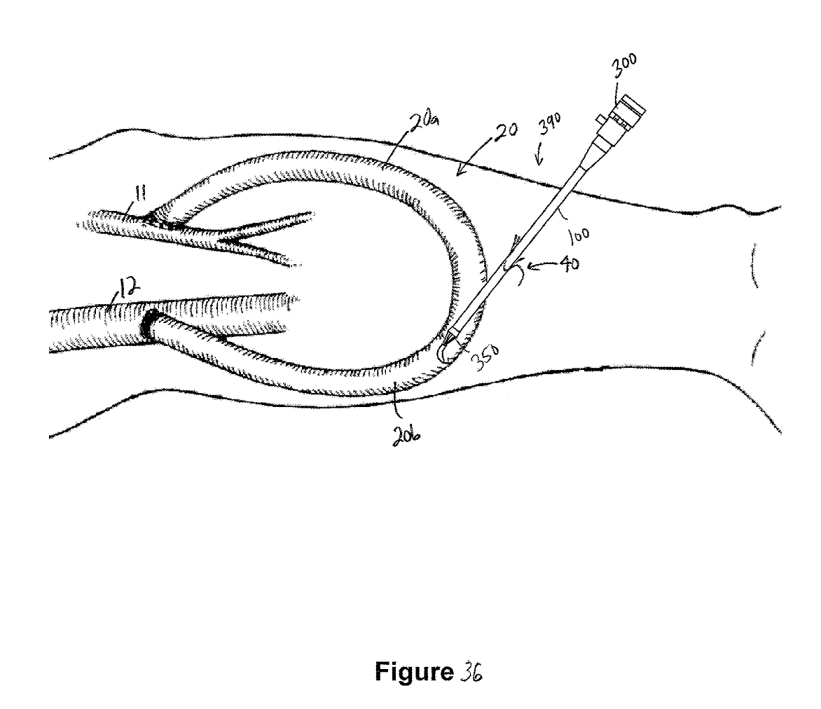

As shown in FIGS. 35-41, vascular sheath 100 and introducer 300 enable controlled percutaneous access to both the venous limb and the arterial limb of an AV graft or fistula with a single puncture, while simultaneously offering the potential to reduce the likelihood of inadvertent loss of access to the AV graft or fistula. Referring first to FIG. 35, vascular sheath 100 is shown extending through a single puncture 40 into AV graft 20. In particular, vascular sheath 100 is positioned to provide access to the venous limb 20b of AV graft 20. Namely, proximal end 100a is external the patient and distal end 100b is disposed in venous limb 20b. Moving now to FIG. 36, while the position of sheath 100 in venous limb 20b is maintained, introducer 300, with expandable member 350 in the unexpanded position, is axially inserted and advanced through vascular sheath 100 until tip 358 and expandable member 350 emerge from sheath distal end 100b into venous limb 20b, which occurs simultaneous with axial engagement of introducer base 211 and sheath proximal end 100a and coupling of sheath 100 and introducer 200 via mating engagement of arms 212 and annular recess 103.

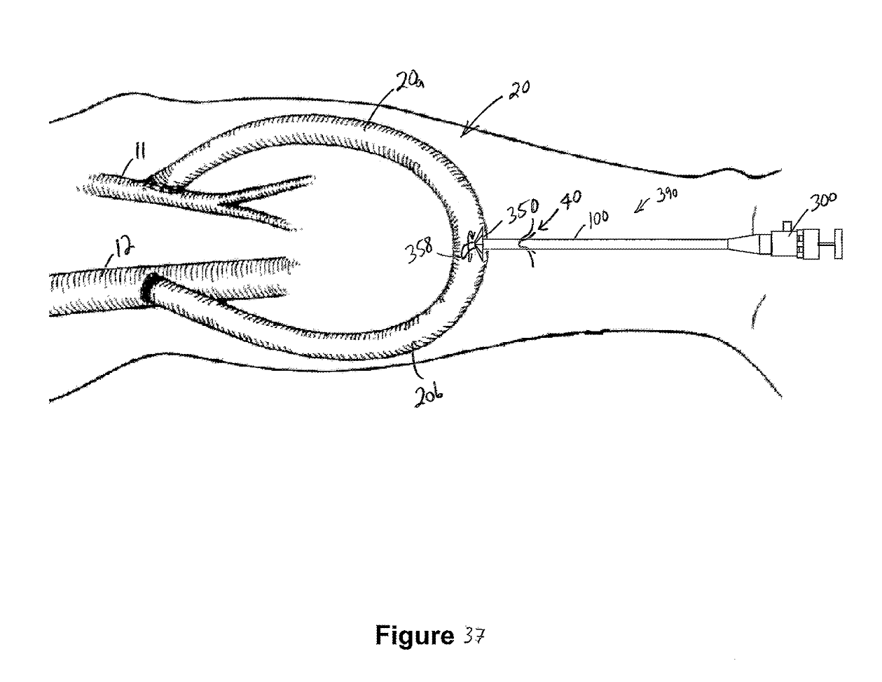

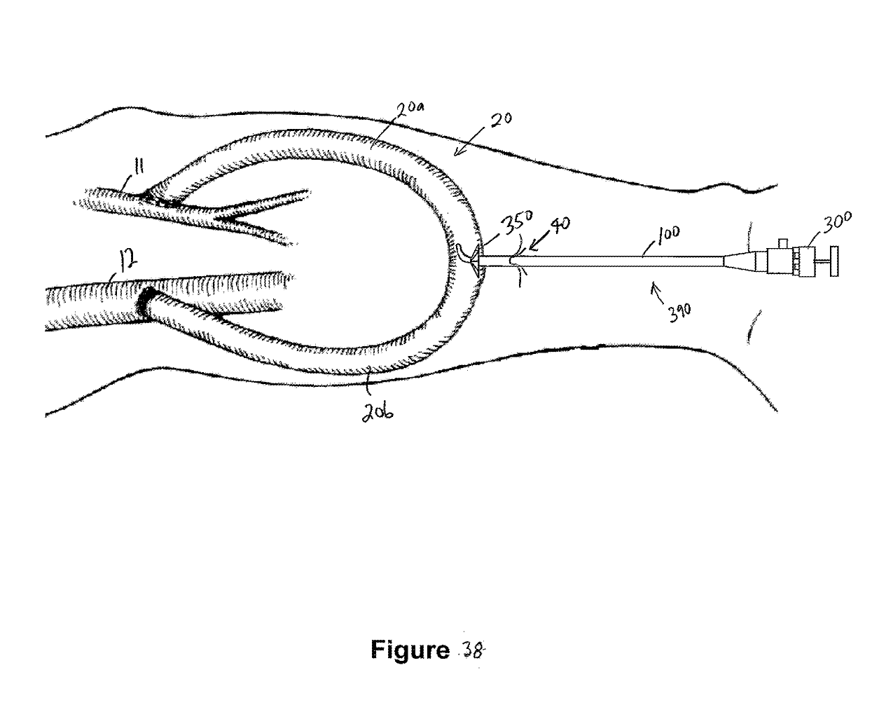

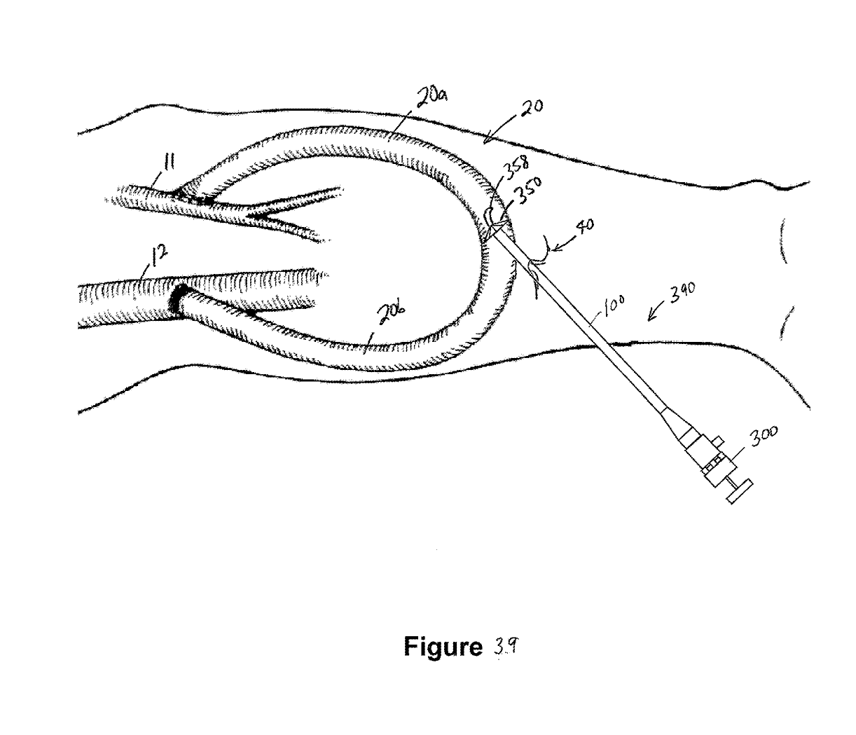

As shown in FIGS. 37 and 38, expandable member 350 is transitioned to the expanded position and sheath-introducer assembly 390 is pulled back through puncture 40. However, tip 358 and expandable member 350 are restricted and/or prevented from exiting AV graft 20 by the radially expanded member 350. Specifically, in the radially expanded position, expandable member 350 bears against the inner wall of AV graft 20, thereby restricting and/or preventing expandable member 350 and tip 358 from inadvertently exiting AV graft 20 and puncture 40. Engagement of expandable member 350 and the inner wall of AV graft 20 is detected by the operator as an increase in resistance to the retraction of sheath-introducer assembly 390 from puncture 40. Next, while maintaining expandable member 350 in the expanded position, tip 358 is rotated about axes 105, 305 such that tip 358 is pointed towards the arterial limb 20a as shown in FIG. 39. Marker 225 on head 321 may be used by the operator to determine the orientation of tip 358. In general, tip 358 may be rotated by rotating introducer 300 within sheath 100 or rotating the entire sheath-introducer assembly 390.

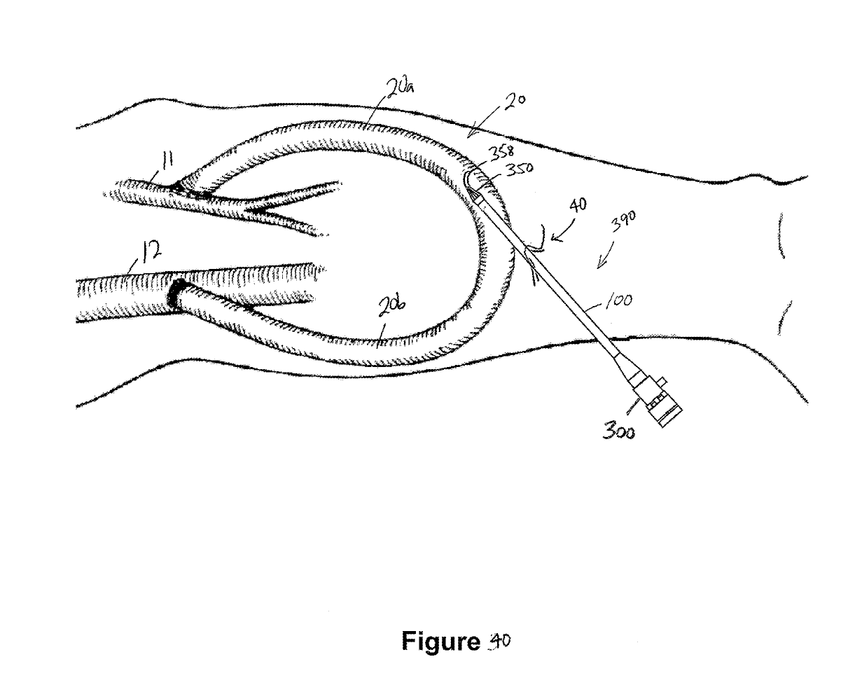

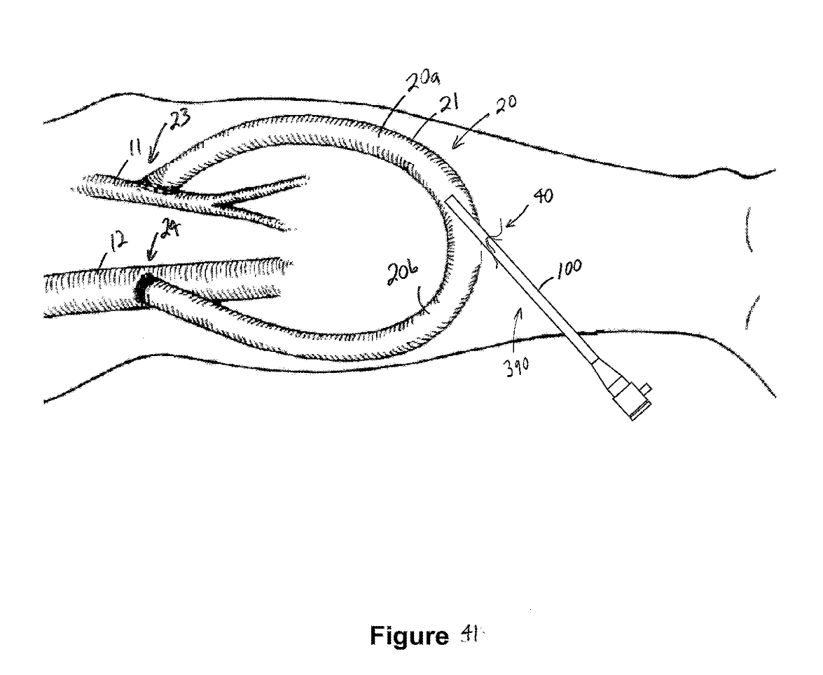

Moving now to FIG. 40, with tip 358 pointing into and generally aligned with venous limb 20b, sheath-introducer assembly 390 is pushed through puncture 40 and AV graft 20 and expandable member 350 is transitioned to the unexpanded position. Accordingly, tip 358 and sheath distal end 100b are urged into arterial limb 20a. By aligning tip 358 with arterial limb 20a prior to urging sheath-introducer assembly 390 further into AV graft 20, tip 358 provides a guide to ensure sheath-introducer assembly 390 advances in the desired direction. With sheath distal end 100b sufficiently positioned to provide access to arterial limb 20a, sheath 100 is maintained in position as introducer 300 is decoupled and removed from sheath 100 as shown in FIG. 41. In the manner described, introducer 300 is used to percutaneously adjust the position of vascular sheath 100, through a single puncture 40, between one position providing access to venous limb 20b and another position providing access to arterial limb 20a. It should be appreciated, that the same basic procedure may also be used to reposition vascular sheath 100 from a position providing access to arterial limb 20a to a position providing access to venous limb 20b through single puncture 40.

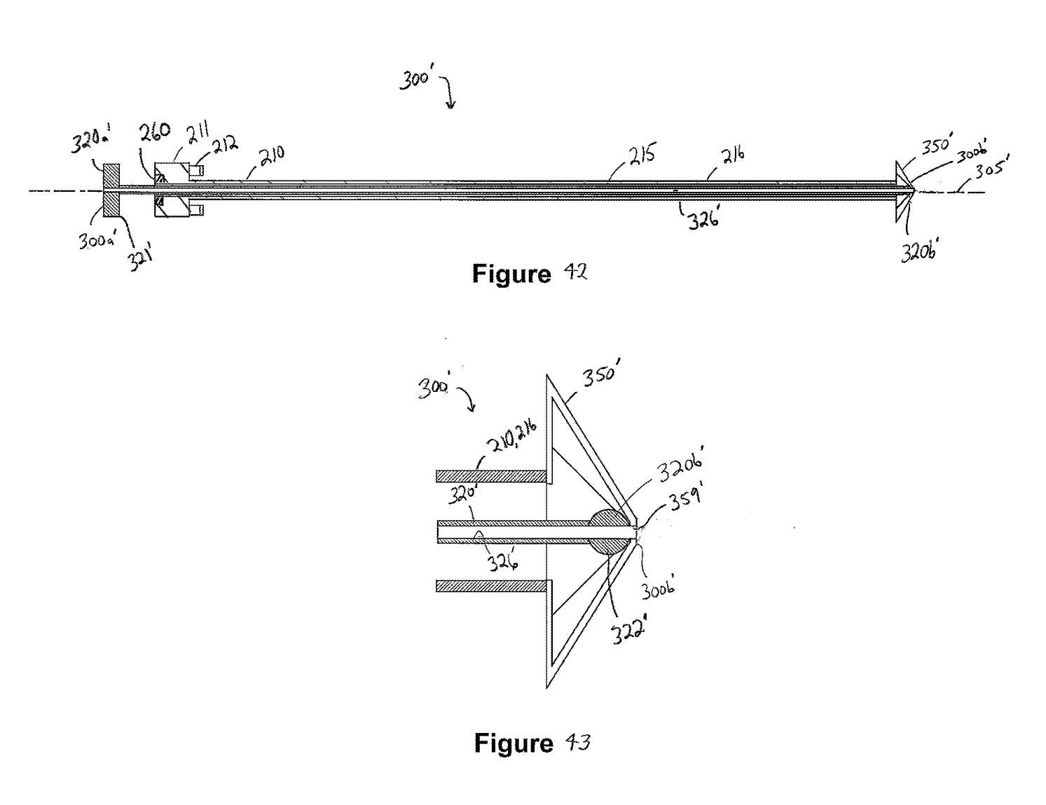

In the embodiment of introducer 300 previously described and shown in FIGS. 28-30, stylet 320 is an elongate solid structure between ends 320a, 320b, distal end 350b of expandable member 350 is occluded, and tip 358 is a solid structure. Thus, introducer 300 is not adapted to accept a guide wire for initial positioning and/or repositioning of vascular sheath 100 within the AV graft 20 through puncture 40. Thus, sheath 100 may be initially positioned in the AV graft 20 as shown in FIG. 35 using a conventional introducer that is passed into the patient over a guide wire, and then introducer 300 may be employed to reposition sheath 100 between the venous and arterial limbs 20b, a. However, referring now to FIGS. 42 and 43, another embodiment of an introducer 300′ suitable for use with a guide wire for the initial placement and/or repositioning of a vascular sheath (e.g., sheath 100) within an AV graft or fistula (e.g., AV graft 20) is shown. Introducer 300′ is substantially the same as introducer 300 previously described with a few exceptions—stylet 320′ of introducer 300′ includes an elongate throughbore 326′ extending axially between ends 320a′, 320b′, stylet 320′ does not include a tapered curved tip (e.g., tip 358), and expandable member 350′ includes a throughbore 359′ extending axially therethrough and aligned with throughbore 326′. Specifically, introducer 300′ has a central or longitudinal axis 305′, a first or proximal end 300a′, and a second or distal end 300b′. In addition, introducer 300′ includes a radially outer housing 210 as previously described, a radially inner stylet 320′ moveably disposed in housing 210, and an expandable member 350′ disposed about stylet 320′ at end 300b′. Expandable member 350′ is substantially the same as expandable member 350 previously described except that expandable member 350′ includes throughbore 359′ and does not include a curved tapered tip (e.g., does not include tip 358). Further, stylet 320′ has a first or proximal end 320a′, a second or distal end 320b′ disposed in expandable member 350′, and includes an enlarged actuation head 321′ at proximal end 320a′, smooth, rounded engagement member 322′ at distal end 320b′, and an elongate, tubular shaft 323′ extending between head 321′ and member 322′. Throughbore 326′ extends through head 321′, shaft 323′, and tip 322′ between ends 320a′, 320b′. Expandable member 350 is disposed about engagement member 322′ and functions as previously described. A locking mechanism 260 as previously described controllably and releasably couples shaft 323′ and housing 310.

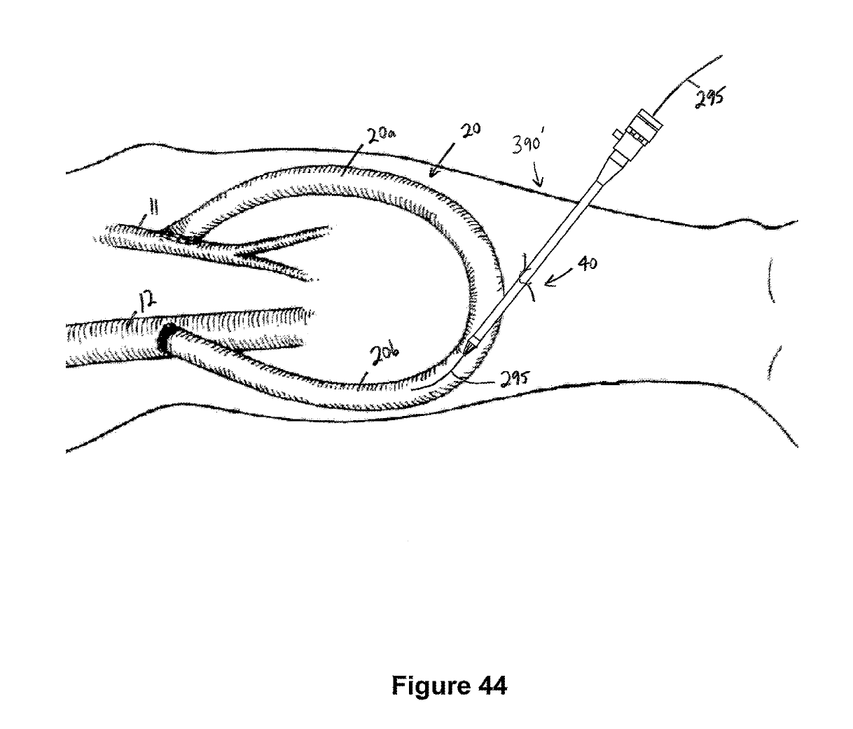

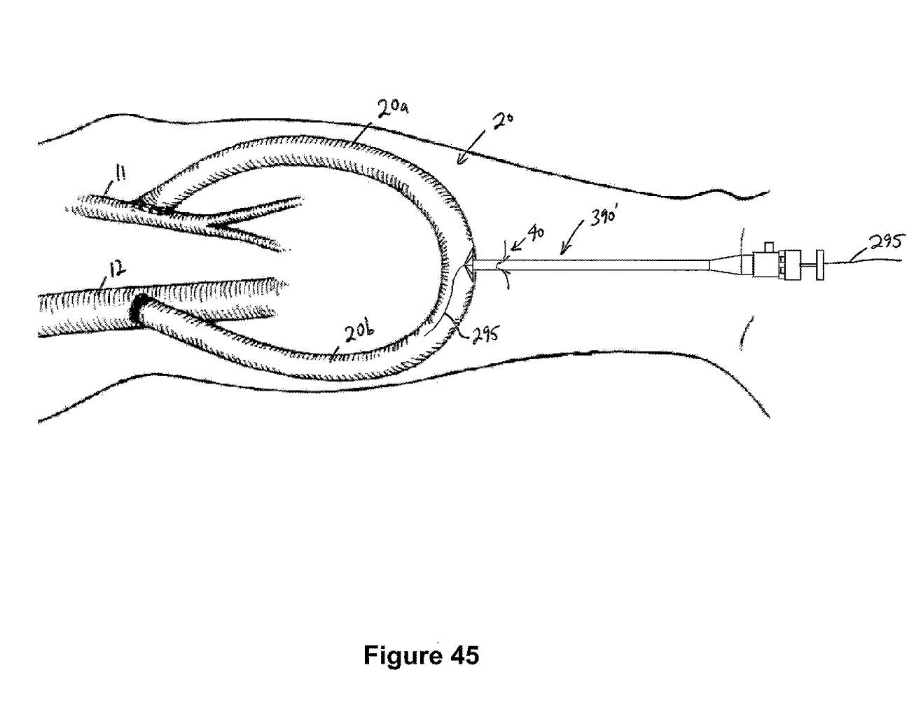

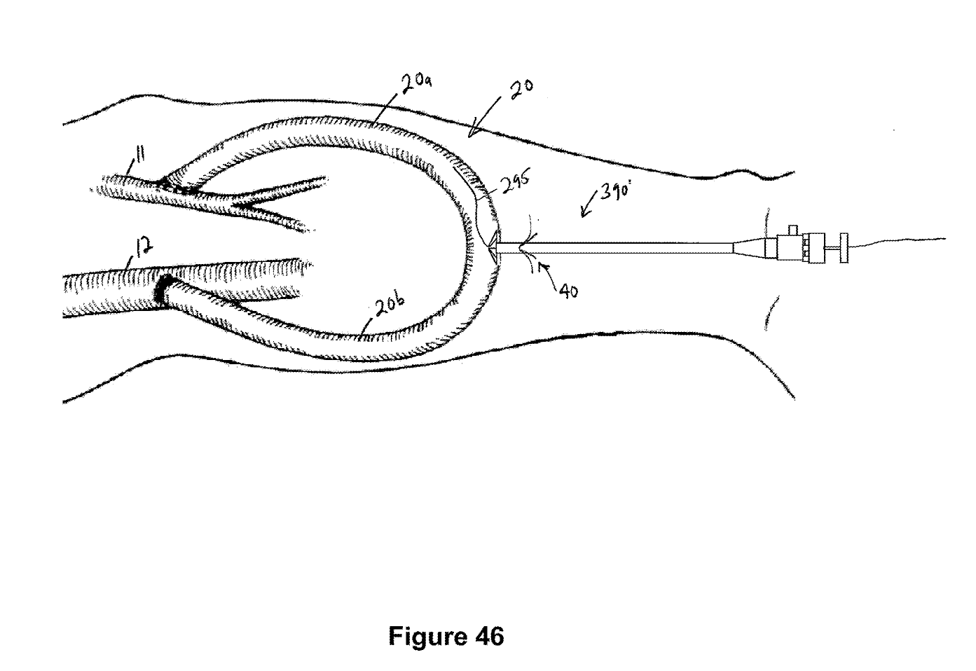

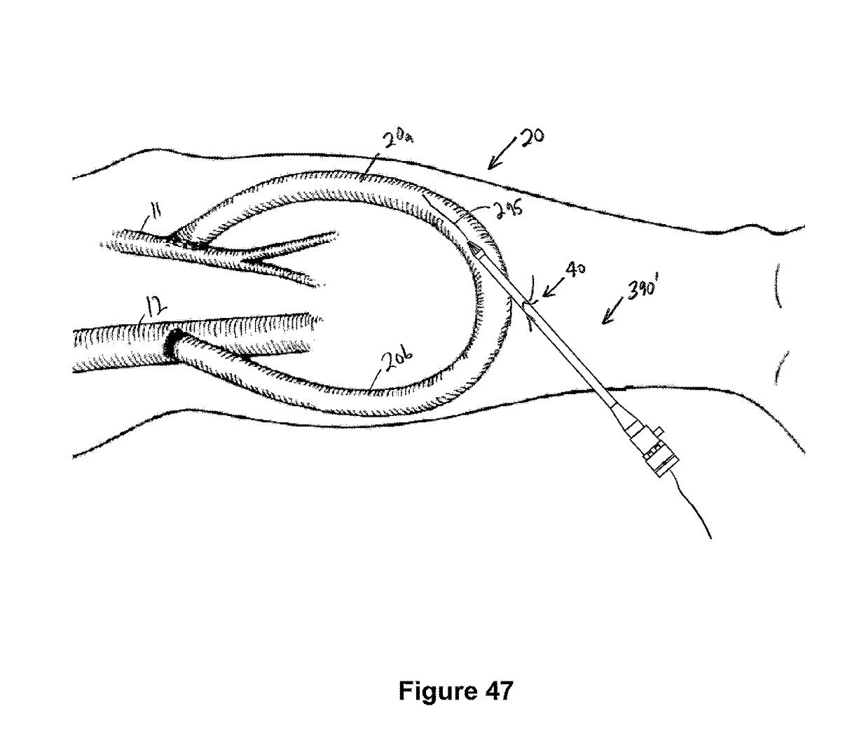

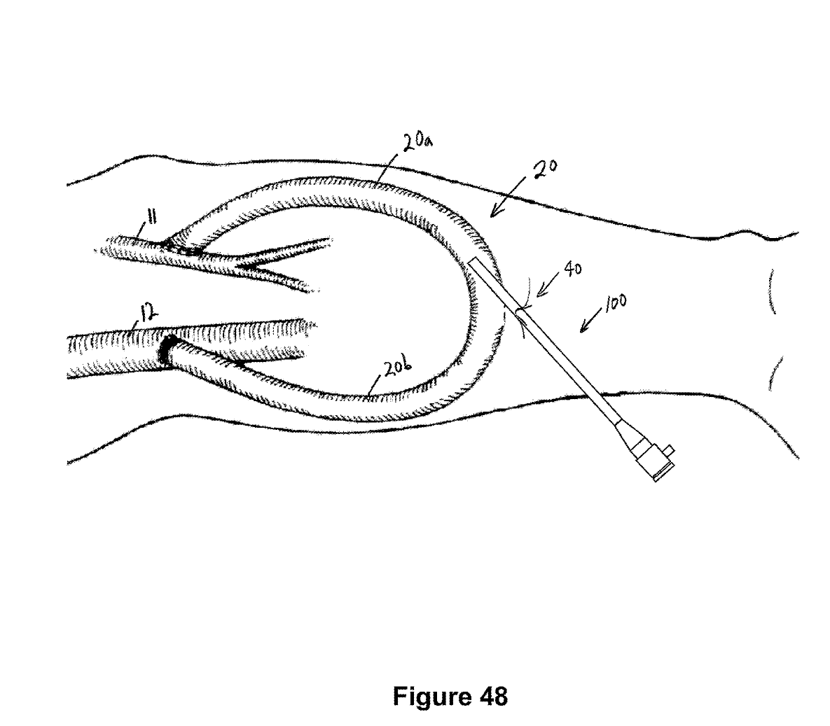

Introducer 300′ is designed for use with a guide wire 295. For example, as shown in FIG. 44, a sheath-introducer assembly 390′ including sheath 100 previously described mounted to introducer 300′, may be passed over a guide wire 295 extending into AV graft 20 to initially position the vascular sheath 100 within the AV graft 20. Further, guide wire 295 may also be employed to reposition vascular sheath 100 from one limb 20a, b to the other limb 20a, b. In particular, with sheath 100 and guide wire 295 in the venous limb 20b of the AV graft 20, introducer 300′ may be advanced over guide wire 295 and loaded coaxially into sheath 100. Then, as shown in FIG. 45, expandable member 350′ may then be transitioned to the expanded position, and then, leaving guide wire 295 in place (extending into the venous limb 20b), sheath-introducer assembly 390′ is pulled back through puncture 40. However, expandable member 350′ restricts and/or prevents assembly 390′ from exiting AV graft 20. Engagement of expandable member 350′ and the inner wall of AV graft 20 is detected by the operator as an increase in resistance to the retraction of sheath-introducer assembly 390′. Next, as shown in FIG. 46, while maintaining expandable member 350′ in the expanded position, guide wire 295 is carefully pulled back under fluoroscopic guidance until its distal tip reaches introducer distal end 300b′, and then guide wire 295 is manipulated towards the arterial limb 20a of the AV graft 20. Moving now to FIG. 47, guide wire 295 is extended into the arterial limb 20a, expandable member 350′ is transitioned to the unexpanded position, and assembly 390′ is advanced over wire 295 into the arterial limb 20a. With sheath distal end 100b sufficiently positioned to provide access to arterial limb 20a, sheath 100 is maintained in position as introducer 300′ is decoupled and removed from sheath 100 as shown in FIG. 48.

Although this embodiment of introducer 300′ includes eliminates curved, tapered tip 358′, and instead, relies on guide wire 295 to align and advance introducer 300′ into the desired limb (e.g., arterial limb 20a or venous limb 20b), in other embodiments, the introducer suitable for use with a guide wire (e.g., introducer 200′) may still include a curved, tapered tip (e.g., tip 358′). Such a curved, tapered tip may be used in conjunction with the guide wire (e.g., guide wire 295) to guide the introducer into the desired limb of the AV graft or fistula.

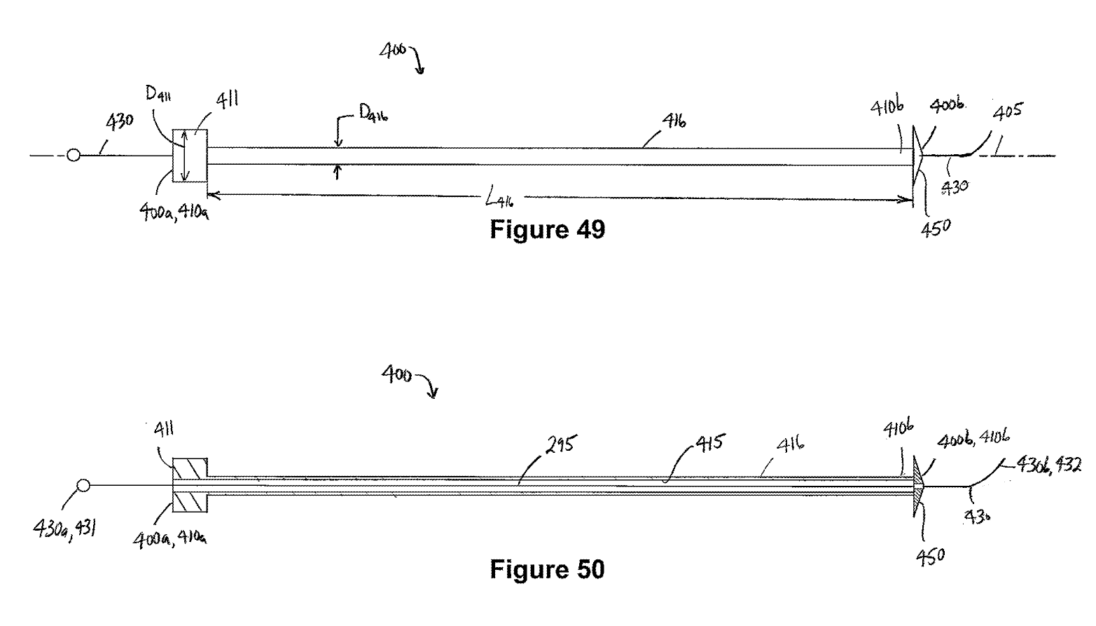

Referring now to FIGS. 49 and 50, an embodiment of a vascular sheath introducer 400 is shown. In general, introducer 400 is a device used to position a vascular sheath, such as vascular sheath 100 previously described, into a vessel, AV graft, or AV fistula, and manipulate the vascular sheath within the vessel, AV graft, or AV fistula. As will be described in more detail below, introducer 400 enables a vascular sheath (e.g., vascular sheath 100) to be pivoted or swiveled back and forth within the lumen of an AV graft or fistula between the venous limb and arterial limb as many times as necessary, while reducing likelihood of inadvertent access loss and associated problems. As a result, embodiments of introducer 400 allow the operator to access both the arterial and venous limb of an AV graft or fistula via a single puncture. Further, embodiments of introducer 400 allow the operator to access an AV graft or AV fistula at any level and work in antegrade and retrograde fashions, via a single puncture.

In this embodiment, introducer 400 has a central or longitudinal axis 405, a first or proximal end 400a, and a second or distal end 400b. Further, introducer 400 comprises a tubular housing 410 and an expandable member 450 coupled to housing 410. A guide wire 430 is slidably and removably disposed in housing 410, however, no stylet is provided within housing 410.

Housing 410 is similar to housing 210 previously described. Namely, housing 410 extends axially between a first or proximal end 410a defining introducer end 400a and a second or distal end 410b coupled to expandable member 450. In addition, housing 410 includes a base 411 at proximal end 410a and an elongate tubular or sleeve 416 extending from base 411 to end 410b. Sleeve 416 has a length L₄₁₆ measured axially between base 4211 and end 410b that is greater than vascular sheath length L₁₀₀. In this embodiment, base 411 and sleeve 416 are each generally cylindrical. In particular, base 411 has an outer diameter D₄₁₁, and sleeve 416 has an outer diameter D₄₁₆ that is less than diameter D₄₁₁. Housing 410 also includes a central throughbore or passage 415 that extends axially through base 411 and sleeve 416 between ends 410a, b.

Unlike base 211 previously described, in this embodiment, base 411 does not include coupling arms 212 to releasably engage annular recess 103 of vascular sheath 100 previously described to restrict and/or prevent housing 410 from moving axially relative to vascular sheath 100 when sheath 100 is mounted to introducer 400. Rather, in this embodiment, introducer 400 may be moved axially relative to sheath 100 during initial positioning and repositioning of sheath 100. In general, housing 410 may be made of the same or similar materials as housing 210 previously described.

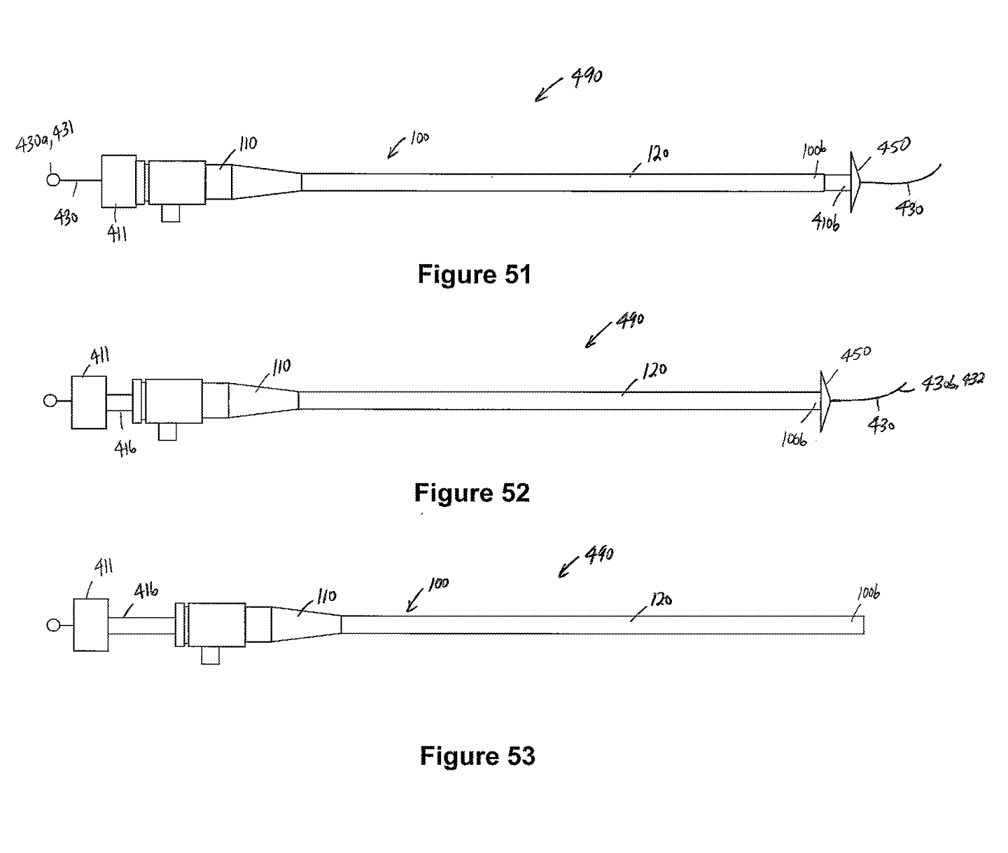

Referring now to FIGS. 49-51, radially expandable member 450 is attached to distal end 410b of housing 410 and defines end 400b of introducer 400. As best shown in FIG. 51, expandable member 450 includes a throughbore 455 extending axially through member 450 and coaxially aligned with passage 415. As will be described in more detail below, in this embodiment, expandable member 450 is a flexible, resilient annular flange or disc that is biased to the radially expanded position shown in FIGS. 49-51.

Referring now to FIGS. 51-53 and 54A-C, vascular sheath 100 previously described is shown coaxially mounted to introducer 400 to form a vascular sheath-introducer assembly 490. To assemble sheath-introducer assembly 490, introducer 400 is inserted and advanced coaxially through sheath bore 101. In particular, introducer end 400b and expandable member 450 are inserted into sheath end 100a and advanced through sheath 100 toward sheath distal end 100b. Introducer 400 may be advanced through sheath 100 until body 411 axially abuts sheath end 100a. Length L₄₁₆ of introducer sleeve 416 is greater than length L₁₀₀ of sheath 100, and thus, when body 411 engages sheath proximal end 100a, introducer distal end 400b and expandable member 450 extend axially from sheath distal end 100b as shown in FIG. 51. Sleeve 416 is preferably sufficiently long to enable expandable member 450 to be axially extended beyond both sheath distal end 100b such that it may transition to the expanded position.

Expandable member 450 may be described as having a radially expanded position in which expandable member 450 is not restricted from expanding radially outward, and an unexpanded or collapsed position in which expandable member 450 is disposed within sheath 100. Specifically, in the radially expanded position, expandable member 450 has an outer diameter D₄₅₀ that is greater than diameter D₁₀₁ of sheath bore 101. However, in the collapsed, unexpanded position, expandable member 450 is disposed within throughbore 101 and is restricted from expanding radially outward by sheath sleeve 120. Thus, in the collapsed, unexpanded position, outer diameter D₄₅₀ of expandable member 450 is the same as inner D₁₀₁ of sheath sleeve 120.

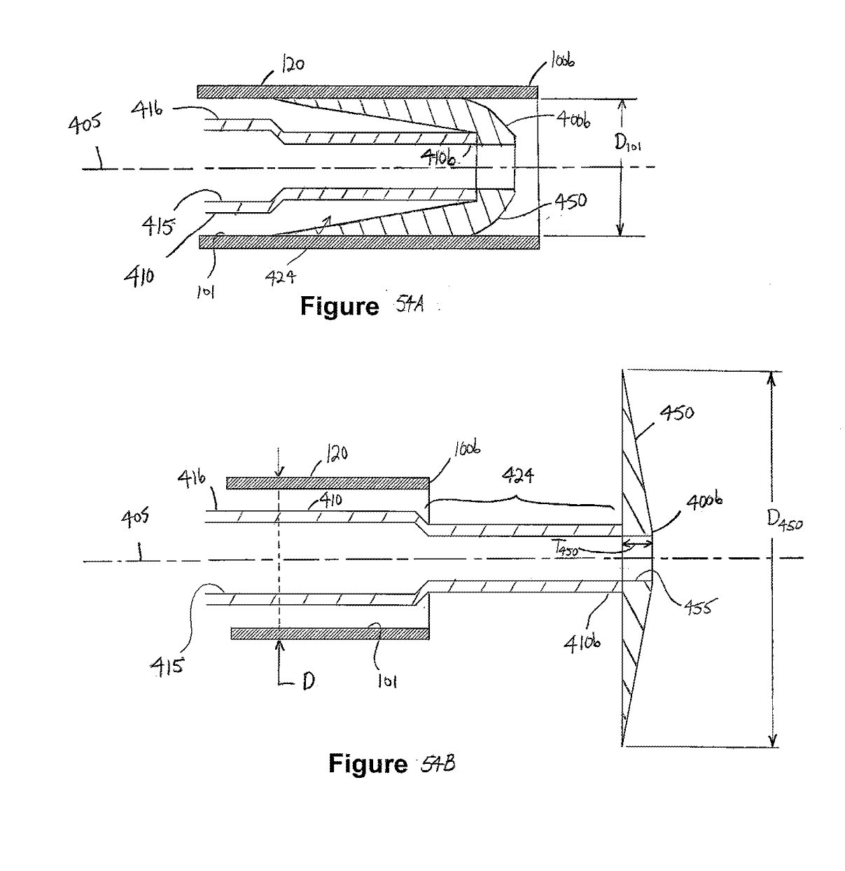

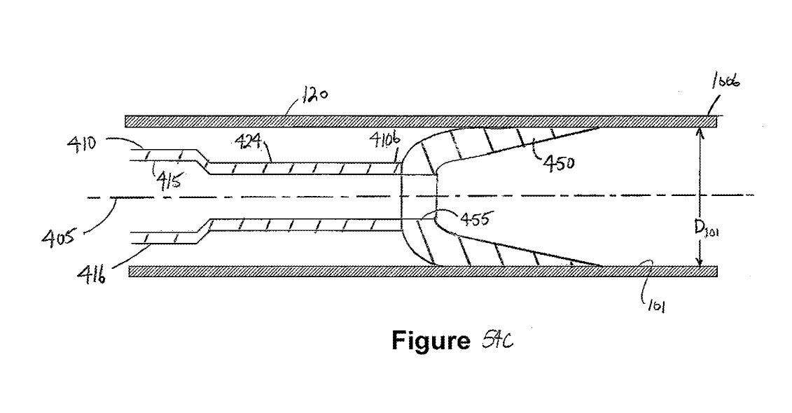

When expandable member 450 extends from sheath 100, expandable member 450 is free to expand radially outward as shown in FIG. 51, however, when expandable member 450 is disposed in sheath 100, expandable member 450 is collapsed to the unexpanded position; when expandable member 450 is disposed within sheath 100, sheath 100 restricts and prevents expandable member 450 from expanding radially outward. For example, in FIG. 54A, expandable member 450 is shown in a collapsed, unexpanded position as expandable member 450 is being urged axially through sheath 100 toward end 100b; in FIG. 54B, expandable member 450 is shown in the radially expanded position extending from sheath 100; and in FIG. 54C, expandable member 450 is shown in a collapsed, unexpanded position as expandable member 450 is being retracted or pulled back into sheath 100 through end 100b. As best shown in FIG. 54A, in this embodiment, housing distal end 410b comprises an annular recess 424 that enhances the radial distance between sheath sleeve 120 and housing sleeve 416 at end 410b, thereby providing sufficient space and clearance for expandable member 450 in the collapsed position when it is being urged through sheath 100 towards distal end 100b. In the manner described, expandable member 450 is controllably collapsed by pulling expandable member 450 into sheath 100 at end 100b, and is expanded by pushing expandable member 450 out of housing 100 at end 100b. It should be appreciated that expandable member 450 is moved axially into and out of sheath 100 via axial actuation of housing 410 relative to sheath 100.

With sheath 100 mounted to introducer 400, introducer 400 may be used to manipulate the position and angular orientation of sheath 100. With introducer 400 sufficiently positioned within sheath 100, guide wire 430 may be inserted and advanced through housing 410 and expandable member 450.

To facilitate such radial expansion and contraction, expandable member 450 preferably has a thickness T₄₅₀ that is substantially less than its expanded diameter D₄₅₀, and further, preferably comprises a biocompatible, resilient, flexible material capable of being repeatedly collapsed and expanded. For example, the expandable member (e.g., expandable member 450) may be made of a flexible polymer or rubber material. In this embodiment, expandable member 450 is biased to the radially expanded position shown in FIGS. 51, 52, and 54B, and must be forced into the collapsed position by urging expandable member 450 into sheath 100. In other words, expandable member 450 is relaxed in the radially expanded position, and unrelaxed and under radially compressive loads in the unexpanded position. Since sheath sleeve 120 prevents expandable member 450 from radially expanding when expandable member 450 is disposed therein, and frictional engagement between expandable member 450 and sheath sleeve 120 restricts and/or prevents inadvertent axial movement of expandable member 450 relative to sheath 100, a locking mechanism (e.g., locking mechanism 260) is not included in this embodiment. However, to aid in control and manipulation, in other embodiments, a locking mechanism (e.g., locking mechanism 260) may be included to releasably lock the housing (e.g., housing 410) to the sheath (e.g., sheath 100).

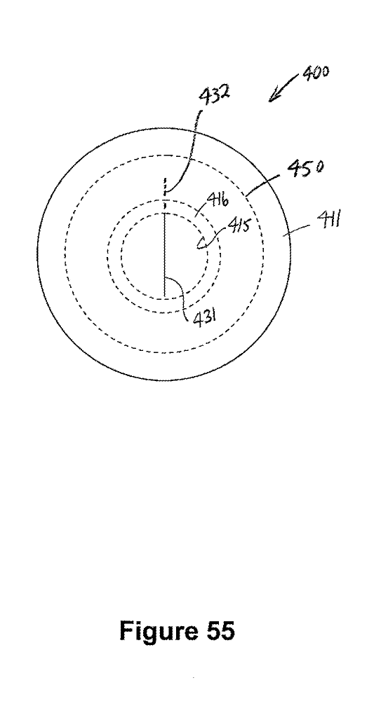

Referring again to FIGS. 50-52, elongate guide member or wire 430 is removably disposed in throughbore 415 and extends axially through housing 410. Guide wire 430 has a proximal end 430a comprising a handle 431 and a distal end 430b comprising a curved tip 432. Guide wire 430 is slidingly disposed in housing 410, and thus, can be moved axially through bore 415 relative to housing 410. Handle 431 is employed to move guide wire 430 and tip 432 relative to housing 410. For example, handle 431 may be axially pulled to withdraw tip 432 into throughbore 415 or axially pushed to extend tip 432 from throughbore 415. In this embodiment, handle 431 is a loop. Guide wire 430 is preferably made of a biocompatible, resilient material that allows curved tip 432 to be elastically deformed to fit through bore 415 and elastically return to its original shape upon exiting bore 415. One example of a suitable material for the guide wire (e.g., guide wire 430) is nitinol. As best shown in FIG. 55, handle 431 is circumferentially aligned with tip 432. Thus, the operator of introducer 400 can utilize handle 431 to determine the orientation of curved tip 432 when curved tip 432 is not in the line of sight (e.g., tip 432 is disposed within the AV graft or fistula).

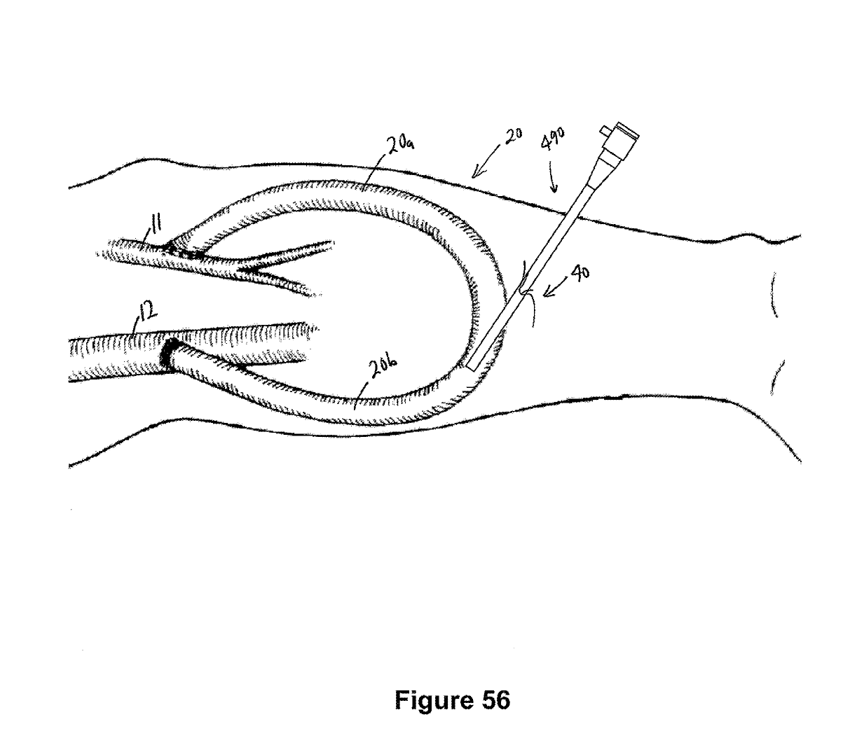

As shown in FIGS. 56-62, vascular sheath 100 and introducer 400 enable controlled percutaneous access to both the arterial limb and the venous limb of an AV graft with a single puncture, while simultaneously offering the potential to reduce the likelihood of inadvertent loss of access to the AV graft. Referring first to FIG. 56, vascular sheath 100 is shown extending through a single puncture 40 into AV graft 20. In particular, vascular sheath 100 is positioned to provide access to the venous limb 20b of AV graft 20. Namely, proximal end 100a is external to the patient and distal end 100b is disposed in venous limb 20b.

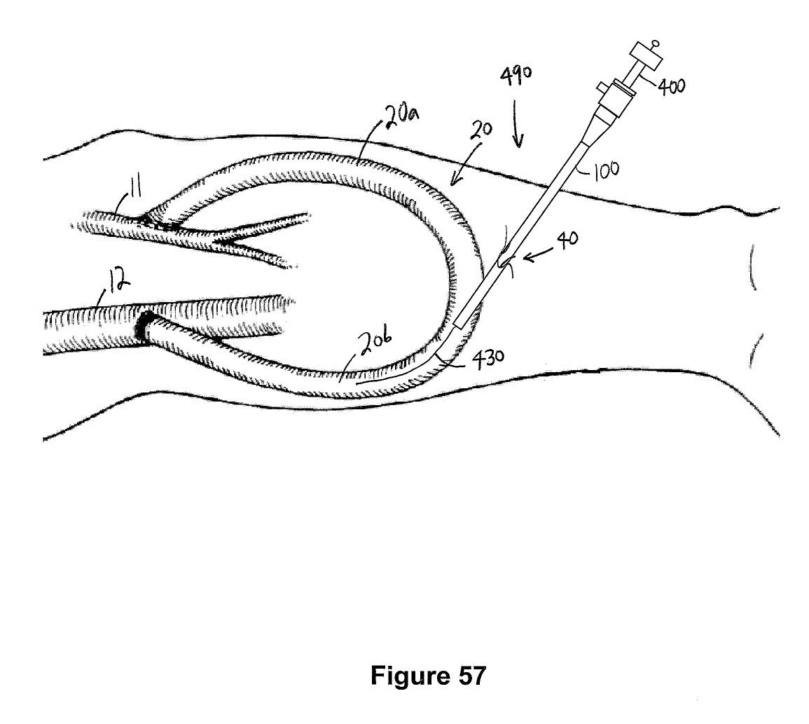

Moving now to FIG. 57, while the position of sheath 100 in venous limb 20b is maintained, guide wire 430 is advanced through sheath 100 into venous limb 20b, and then, introducer 400 is axially advanced over guide wire 430 through vascular sheath 100. During insertion of introducer 400 into sheath 100, expandable member 450 is in the unexpanded position within housing 410.

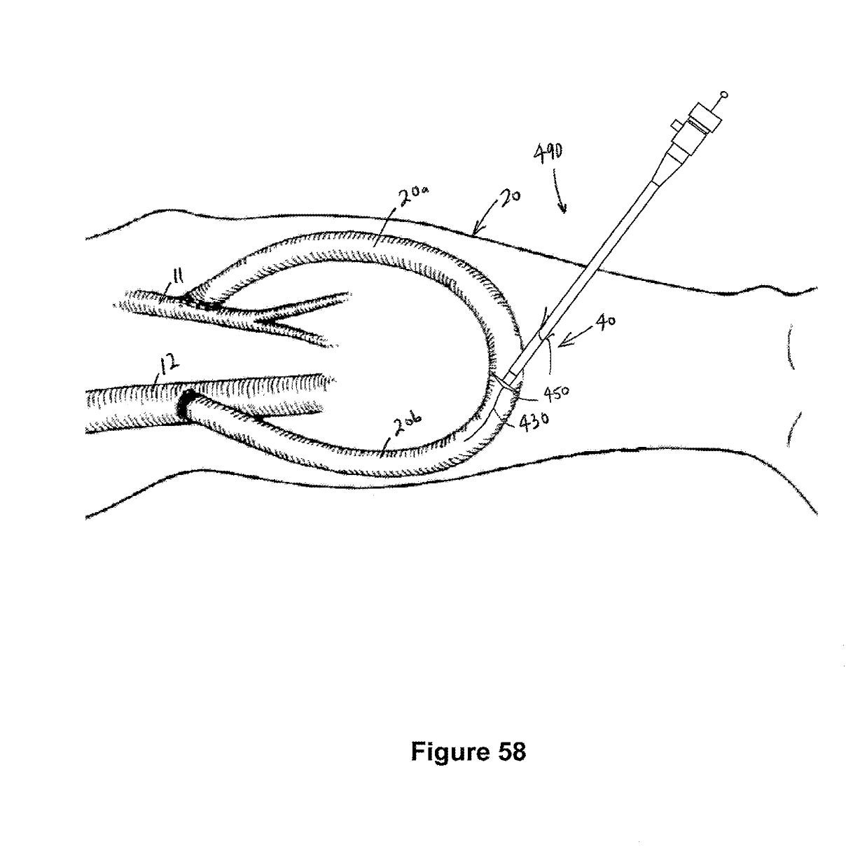

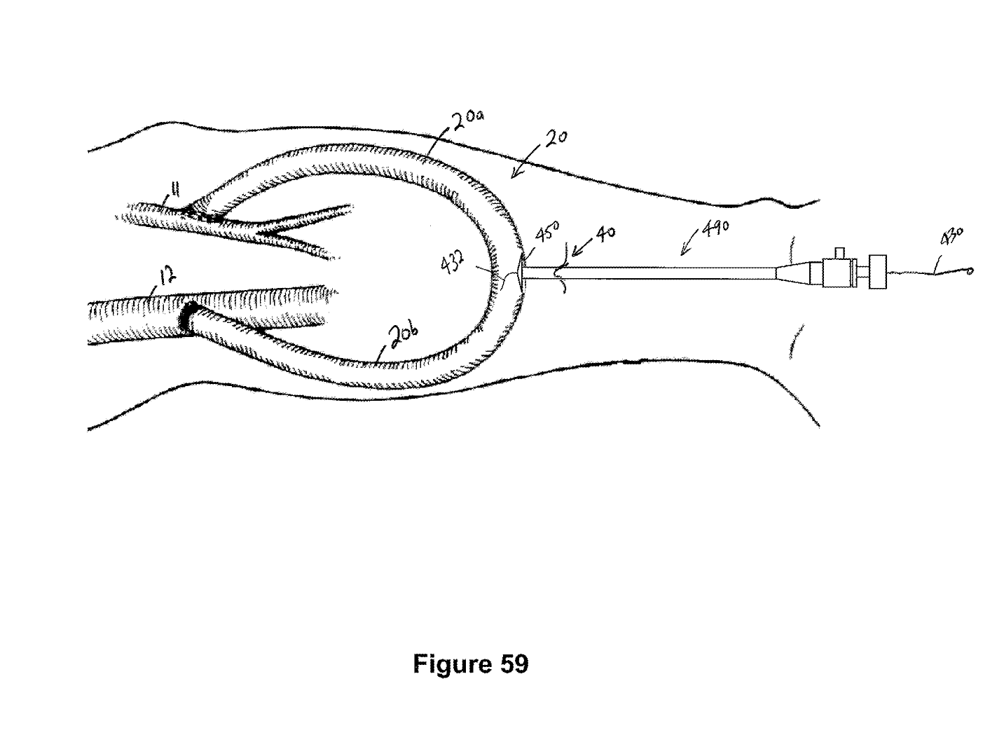

Moving now to FIGS. 58 and 59, expandable member 450 is advanced through sheath 100 until axial engagement of base 411 and sheath proximal end 100a, thereby extending expandable member 450 from sheath distal end 100b and allowing expandable member 450 to transition to its radially expanded position. Base 411 is then pulled back relative to sheath 100 until resistance is felt, indicating expandable member 450 has axially abutted and engaged distal end 100b of sheath 100. Further, sheath-introducer assembly 490 is pull back over guide wire 430 through puncture 40. However, sheath-introducer assembly 490 is restricted and/or prevented from completely exiting AV graft 20 by expandable member 450. Specifically, in the radially expanded position, expandable member 450 bears against the inner wall of AV graft 20, thereby restricting and/or preventing expandable member 450 from inadvertently exiting AV graft 20 and puncture 40. Engagement of expandable member 450 and the inner wall of AV graft 20 is detected by the operator as an increase in resistance to the retraction of sheath-introducer assembly 490 from puncture 40. This step may easily be performed and/or confirmed under fluoroscopic guidance.

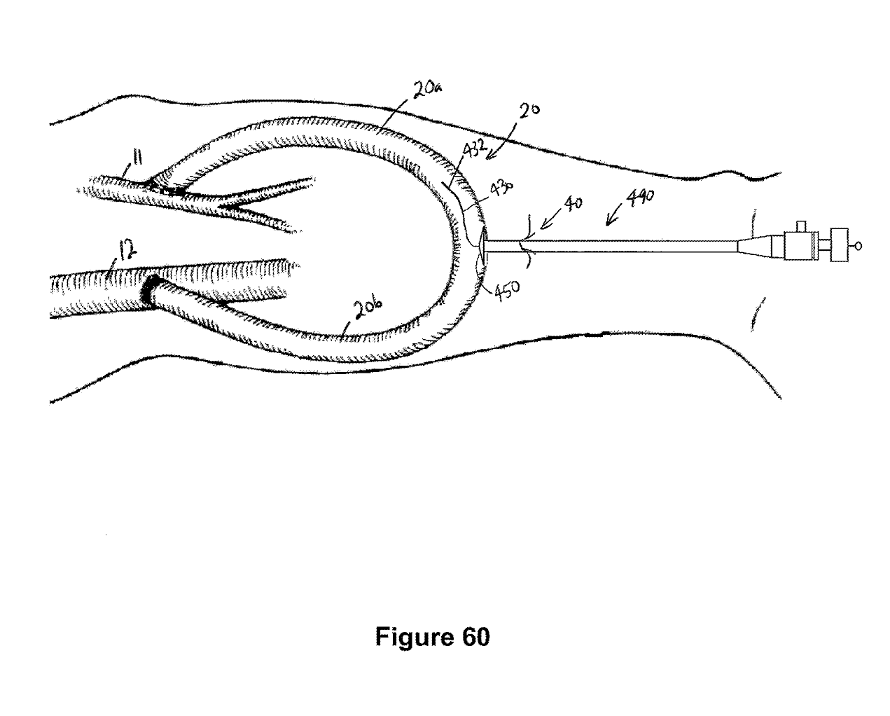

Next, while maintaining expandable member 450 in the expanded position, guide wire 430 is pull back to sheath distal end 100b, redirected under fluoroscopy and advanced from expandable member 450 into the arterial limb 20a of AV graft 20 as shown in FIGS. 59 and 60. Handle 431 may be used by the operator to determine the orientation of wire tip 432. In general, tip 432 may be rotated by rotating handle 431. By aligning tip 432 with venous limb 20b prior to urging sheath-introducer assembly 490 further into AV graft 20, tip 432 provides a guide to ensure sheath-introducer assembly 490 advances in the desired direction.

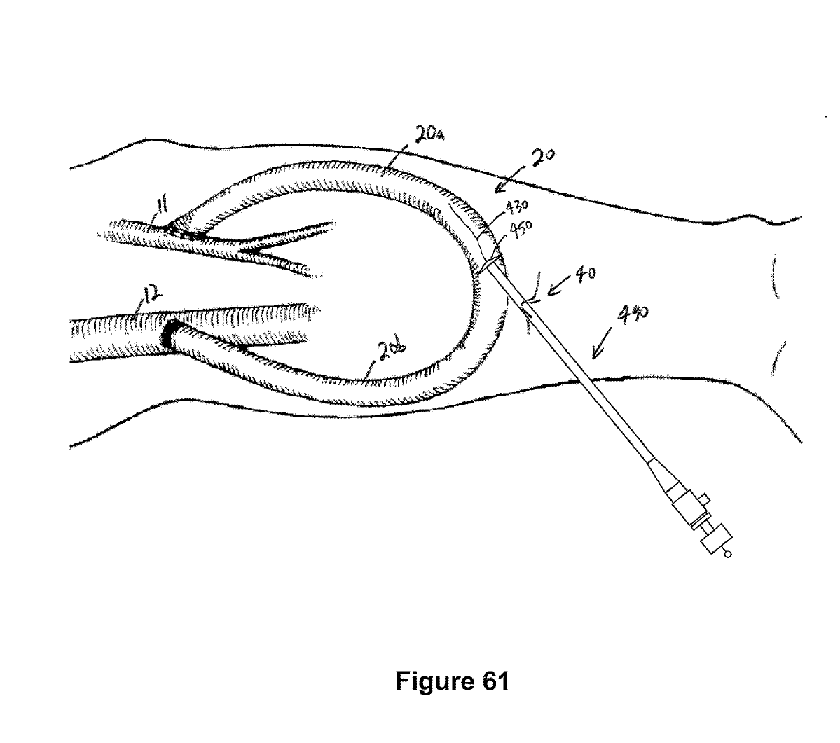

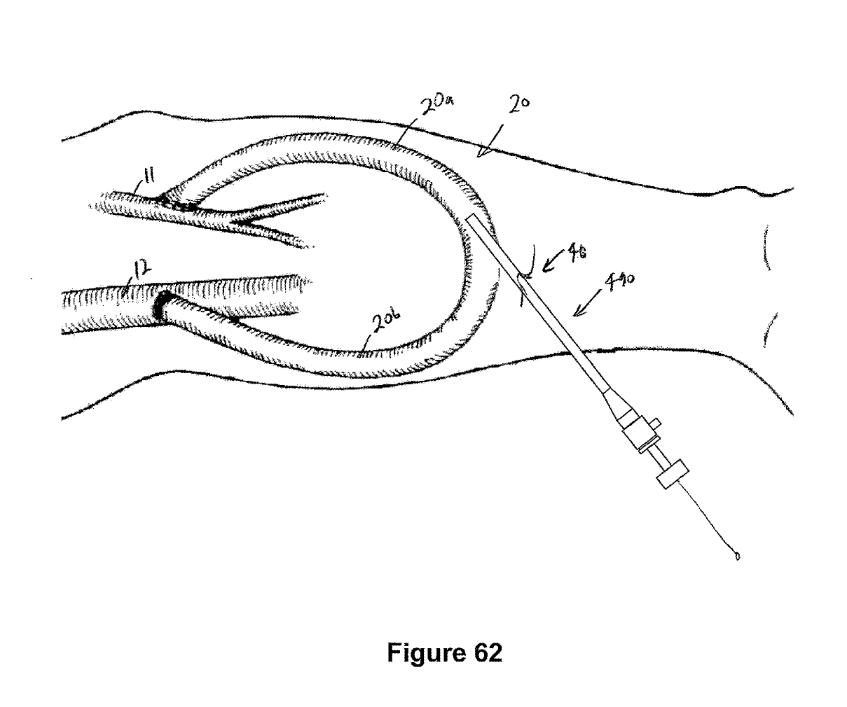

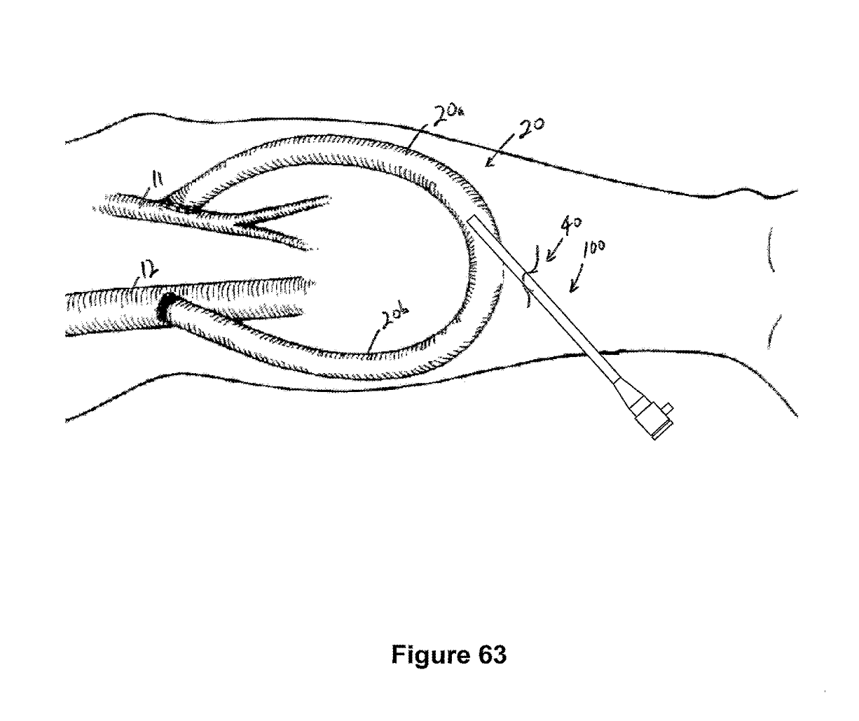

As shown in FIG. 61, sheath-introducer assembly 490 is then advanced over guide wire 430 into arterial limb 20a. In addition, expandable member 450 is transitioned to the unexpanded position by retracting expandable member 450 into sheath 100. With sheath distal end 100b sufficiently positioned to provide access to arterial limb 20a, sheath 100 is maintained in position as introducer 400 is decoupled and removed from sheath 100 as shown in FIG. 62. Although guide wire 430 is shown as being withdrawn with introducer 400 in FIG. 62, in other embodiments, guide wire 430 may be maintained in sheath 100 as introducer 400 is withdrawn from sheath 100, thereby enabling subsequent reinsertion of introducer 400 over guide wire 430 into sheath 100.

In the manner described, introducer 400 is used to percutaneously adjust the position of vascular sheath 100, through a single puncture 40, between one position providing access to venous limb 20b and another position providing access to arterial limb 20a. It should be appreciated, that the same basic procedure may also be used to reposition vascular sheath 100 from a position providing access to arterial limb 20a to a position providing access to venous limb 20b through single puncture 40, as many times as required during the procedure.

Referring now to FIGS. 64-66, an embodiment of a vascular sheath introducer 500 is shown. In general, introducer 500 is a device used to position a vascular sheath, such as vascular sheath 100 previously described, into a vessel, AV graft, or AV fistula, and manipulate the vascular sheath within the vessel, AV graft, or AV fistula. As will be described in more detail below, introducer 500 enables a vascular sheath (e.g., vascular sheath 100) to be pivoted or swiveled back and forth within the lumen of an AV graft or fistula between the venous limb and arterial limb as many times as necessary, while reducing likelihood of inadvertent access loss and associated problems. As a result, embodiments of introducer 500 allow the operator to access both the arterial and venous limb of an AV graft or fistula via a single puncture. Further, embodiments of introducer 500 allow the operator to access an AV graft or AV fistula at any level and work in antegrade and retrograde fashions, via a single puncture.

In this embodiment, introducer 500 has a central or longitudinal axis 505, a first or proximal end 500a, and a second or distal end 500b. In addition, introducer 500 includes a radially outer housing 210 as previously described, a radially inner stylet 520 disposed in throughbore 215 and extending through housing 210. Housing 210 and stylet 520 are each coaxially aligned with longitudinal axis 505. Further, stylet 520 is adapted to move axially relative to housing 210.

Referring still to FIGS. 64-66, stylet 520 extends along axis 505 between a first or proximal end 520a defining introducer end 500a and a second or distal end 520b. In addition, stylet 520 includes an enlarged actuation head 521 at proximal end 520a, an expandable member 550 at distal end 520b, and an elongate tubular 523 extending between head 521 and expendable member 550. Head 521 is fixedly coupled to a proximal end 523a of tubular 523, and expandable member 550 is fixedly attached to a distal end 523b of tubular 523. As will be described in more detail below, axial force may be applied to head 521 by the operator of introducer 500 to move head 521, elongate tubular 523, and expandable member 550 axially relative to housing 210. Thus, elongate tubular 523 functions to transmit axial forces between head 521 and expandable member 550. In this embodiment, a throughbore 525 extends through stylet 520 from proximal end 520a to distal end 520b. Thus, throughbore 525 extends through head 521, tubular 523, and expandable member 550. To aid in positioning introducer 500, an elongate guide member or wire such as guide wire 430 previously described, may be slidingly disposed in throughbore 525 and extend axially from ends 520a, b.

In general, head 521 and tubular 523 may be made of any suitable material(s) including, without limitation, metals (e.g., aluminum) and metal alloys (e.g., stainless steel, nitinol, etc.), polymers (e.g., plastics), composites (e.g., carbon fiber and epoxy matrix composite), or combinations thereof. However, head 521 and tubular 523 are preferably made of biocompatible, rigid or semi-rigid materials capable of transferring axial forces to expandable member 550 without substantial deformation. Examples of suitable materials for head 521 and elongate tubular 523 include, without limitation, metals and metal alloys, composites, and polymers. In this embodiment, head 521 and tubular 523 are monolithically formed, however, in other embodiments, the stylet head (e.g., head 521) and the stylet tubular (e.g., tubular 523) may be separate and distinct components that are coupled together.

Referring now to FIGS. 64-67B, radially expandable member 550 is attached to distal end 523b of shaft 523 and defines end 520b of stylet 520. In this embodiment, expandable member 550 comprises a plurality of circumferentially spaced resilient wires 551. Each wire 551 has a first or proximal end 551a fixed to distal end 523b of tubular 523 and a second or distal end 551b comprising a rounded or blunt tip 552. Tips 552 are blunted to reduce the likelihood of piercing, cutting, or otherwise damaging the AV graft, fistula, or surrounding tissue when expandable member 550 is transitioned to the expanded position within an AV graft or fistula. In this embodiment, four uniformly circumferentially spaced resilient wires 551 are provided, however, in general, any suitable number of resilient wires (e.g. wires 551) may be included, and further, the wires may be uniformly or non-uniformly circumferentially spaced. In this embodiment, each wire 551 is biased such that it curves radially outward and axially backward toward housing end 210b moving from end 551a to end 551b. Between ends 551a, b, each wire 551 is preferably curved or bent through an angle greater than 90°, and more preferably greater than 120°.

In this embodiment, each wire 551 is biased to the expanded position shown in FIGS. 65, 66, and 67B. In the radially expanded position, expandable member 550 has an outer diameter D₅₅₀ that is greater than outer diameter D₂₁₆ of housing sleeve 216. However, in the collapsed, unexpanded position, expandable member 550 (and each wire 551) is disposed within throughbore 215 and is restricted from expanding radially outward by sleeve 216. Thus, in the collapsed, unexpanded position, outer diameter D₅₅₀ of expandable member 550 is the same as inner D₂₁₅ of housing sleeve 216. Thus, expandable member 550 is controllably collapsed by pulling expandable member 550 into housing 210 at end 210b, and is expanded by pushing expandable member 550 out of housing 210 at end 210b. It should be appreciated that expandable member 550 is moved axially into and out of housing 210 via axial actuation of head 521 and elongate tubular 523.

To facilitate such radial expansion and contraction, each wire 551 preferably comprises a biocompatible, resilient material capable of being repeatedly transitioned between the expanded and unexpanded positions. For example, the wires (e.g., wires 551) may be made of nitinol. As previously described, in this embodiment, expandable member 550 is biased to the radially expanded position shown in FIGS. 65, 66, and 67B, and must be forced into the collapsed position by urging expandable member 550 into housing 210. In other words, expandable member 550 is relaxed in the radially expanded position, and unrelaxed and under radially compressive loads in the unexpanded position. Since housing sleeve 216 prevents expandable member 550 from radially expanding when expandable member 550 is disposed therein, and frictional engagement between expandable member 550 and housing sleeve 216 restricts and/or prevents inadvertent axial movement of expandable member 550 relative to housing 210, a locking mechanism (e.g., locking mechanism 260) may not be necessary in this embodiment.

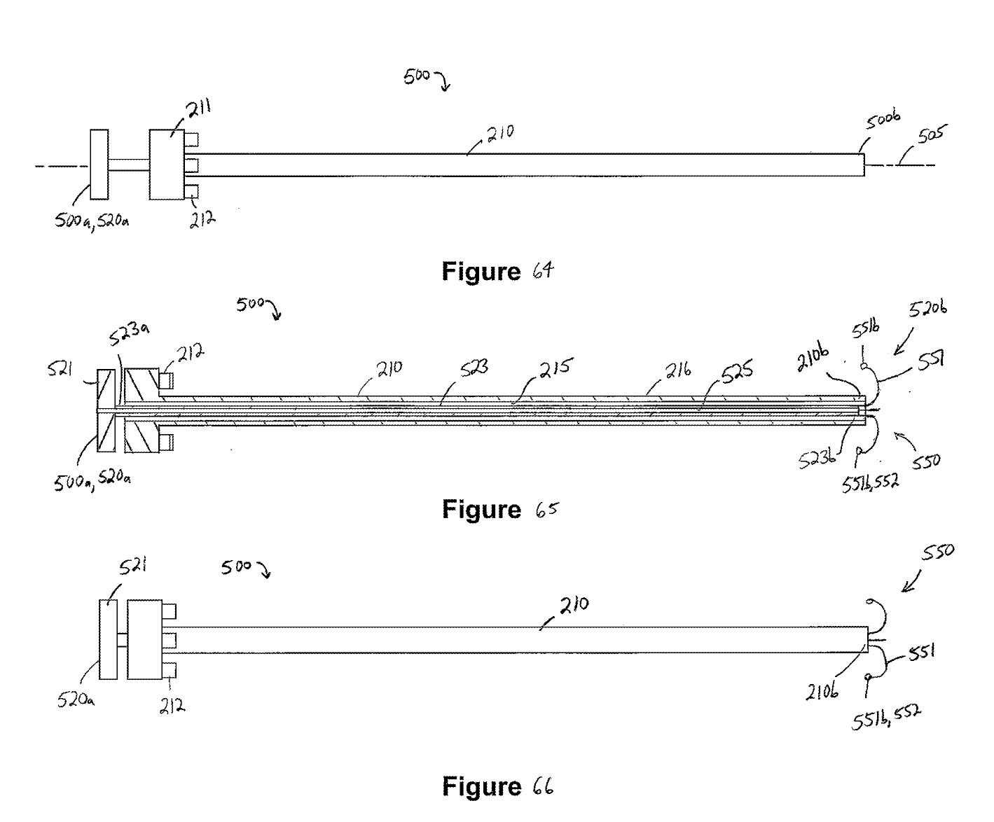

If expandable member 550 is pushed completely through housing 210, expandable member 550 will transition to the expanded position shown in FIGS. 65, 66, and 67B as sleeve 216 will no longer prevent expandable member 550 from radially expanding. Thus, to maintain expandable member 550 in the collapsed position shown in FIG. 67A, expandable member 550 should be maintained within sleeve 216. Once expandable member 550 extends from housing 210 and radially expands, it may be collapsed by simply withdrawing expandable member 550 back into housing throughbore 215 at end 210b.

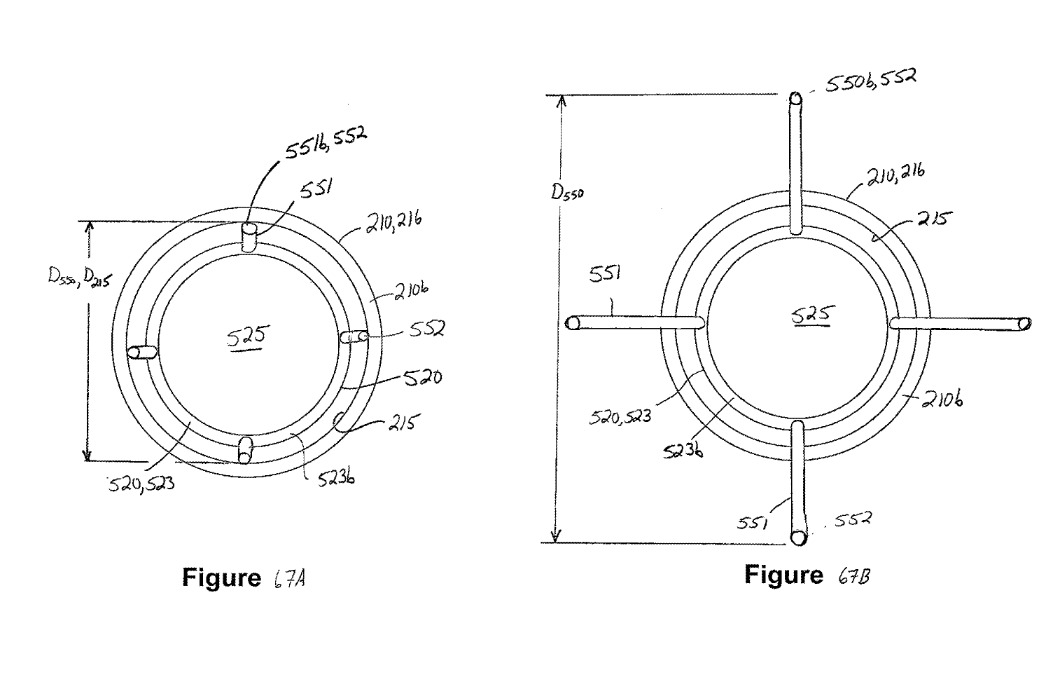

Referring now to FIGS. 68 and 69, vascular sheath 100 previously described is shown coaxially mounted to introducer 500 to form a vascular sheath-introducer assembly 590. With expandable member 550 in the collapsed position within sleeve 216, introducer 500 is axially inserted into sheath 100 at end 100a and axially advanced through sheath 100 toward end 100b. Introducer 500, including expandable member 550, and sheath 100 are sized and configured such that introducer 500 passes through sheath 100, until body 211 axially abuts sheath end 100a simultaneous with positive engagement of lips 213 and annular recess 103. In particular, sleeve outer diameter D₂₁₆ is less than sheath inner diameter D₁₀₁. Sleeve length L₂₁₆ may be the same, less than, or greater than sheath length L₁₀₀, as long as stylet 520 is sufficiently long to enable expandable member 550 to be axially extended beyond both housing distal end 210b and sheath distal end 100b such that it may transition to the expanded position (housing 210 functions to restrict expandable member 550 from radially expanding when expandable member 550 is disposed in housing 210; and sheath sleeve 120 functions to restrict expandable member 550 from radially expanding when expandable member 550 extends from housing 210 but is disposed within sheath 100). As end 100a moves axially into contact with distal ends 212b of arms 212, ends 212b are urged radially outward relative to ends 212a to allow end 100a to pass between arms 212. As end 100a passes between arms 212, lips 213 slidingly engage the outer surface of body 110 and then snap radially inward when aligned with recess 103, thereby releasably locking sheath 100 to introducer 200. With sheath 100 mounted to introducer 500, introducer 500 may be used to manipulate the position and angular orientation of sheath 100. With introducer 500 sufficiently positioned within sheath 100, guide wire 430 may be inserted and advanced through stylet 520. To advance curved tip 432 through stylet bore 525, tip 432 may flex and be urged radially inward by the inner surface of stylet 520.

Vascular sheath 100 and introducer 500 enable controlled percutaneous access to both the arterial limb and the venous limb of an AV graft with a single puncture, while simultaneously offering the potential to reduce the likelihood of inadvertent loss of access to the AV graft. Specifically, introducer 500 is employed to manipulate sheath 100 in substantially the same manner as introducer 400 previously described. Namely, with sheath 100 providing access to the desired limb (e.g., arterial limb 20a or venous limb 20b), introducer 500 is axially inserted and advanced through vascular sheath 100 until axial engagement of introducer base 211 and sheath proximal end 100a and coupling of sheath 100 and introducer 200 via mating engagement of arms 212 and annular recess 103. During insertion of introducer 400 into sheath 100, expandable member 550 is preferably in the unexpanded position within housing 210. Next, stylet head 521 is pushed axially toward base 211 until expandable member 550 extends from both ends 100b, 210b and automatically transitions to the expanded position. Then, sheath-introducer assembly 590 is being pull back through puncture 40, however, sheath-introducer assembly 590 is restricted and/or prevented from completely exiting AV graft 20 by expandable member 550. Specifically, in the radially expanded position, expandable member 550 bears against the inner wall of the AV graft or fistula (e.g., AV graft 20), thereby restricting and/or preventing expandable member 550 from inadvertently exiting the AV graft or fistula. Engagement of expandable member 550 and the inner wall of the AV graft of fistula is detected by the operator as an increase in resistance to the refraction of sheath-introducer assembly 590 assembly from the puncture (e.g., puncture 40). Next, while maintaining expandable member 550 in the expanded position, guide wire 430 is pull back to sheath distal end 100b, redirected and advanced from expandable member 550 into the opposite limb of the AV graft or fistula. Handle 431 may be used by the operator to determine the orientation of wire tip 432. In general, tip 432 may be rotated by rotating guide wire 430 with handle 431. By aligning tip 432 with the desired limb prior to urging sheath-introducer assembly 590 further into the AV graft 20 or fistula, tip 432 provides a guide to ensure sheath-introducer assembly 490 advances in the desired direction. Sheath-introducer assembly 590 is then advanced over wire 430 into the desired limb. In addition, expandable member 550 is transitioned to the unexpanded position by expandable member 550 into sheath 100 via head 521. With sheath distal end 100b sufficiently positioned to provide access to the desired limb, sheath 100 is maintained in position as and guide wire 430 is withdrawn from introducer 500, and introducer 500 is decoupled and removed from sheath 100. In the manner described, introducer 500 is used to percutaneously adjust the position of vascular sheath 100, through a single puncture, between one position providing access to arterial limb and another position providing access to venous limb. It should be appreciated, that the same basic procedure may also be used to reposition vascular sheath 100 from a position providing access to venous limb to a position providing access to arterial limb through a single puncture.

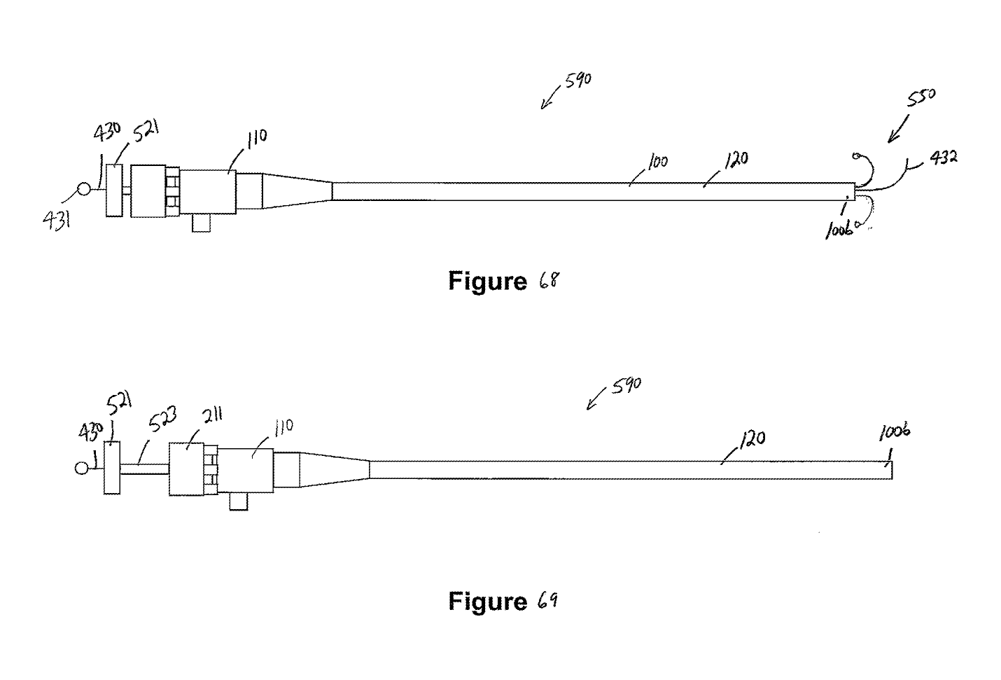

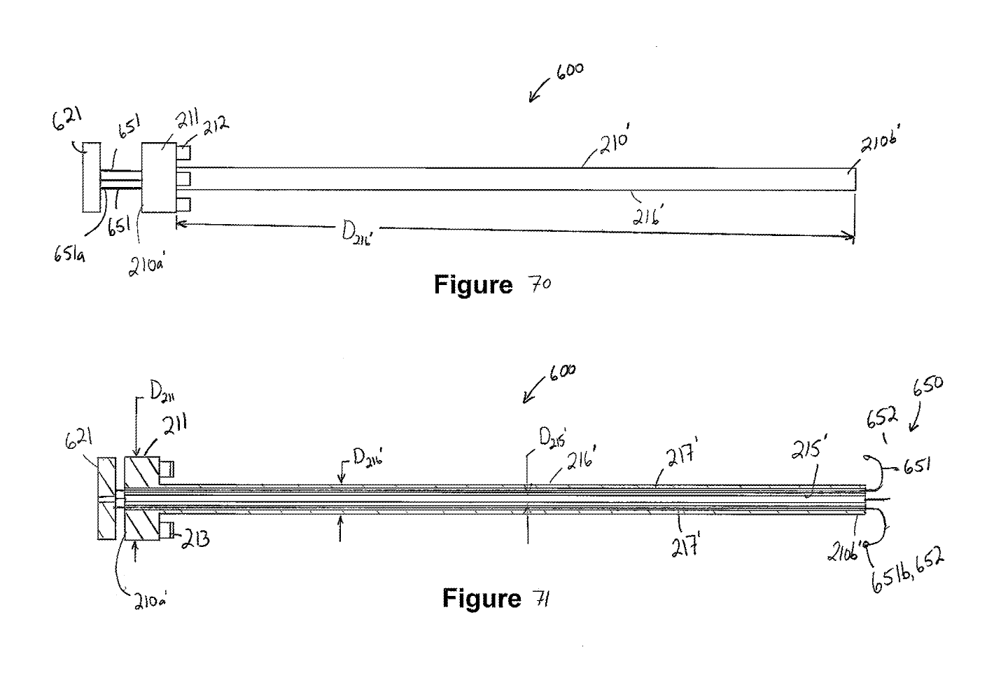

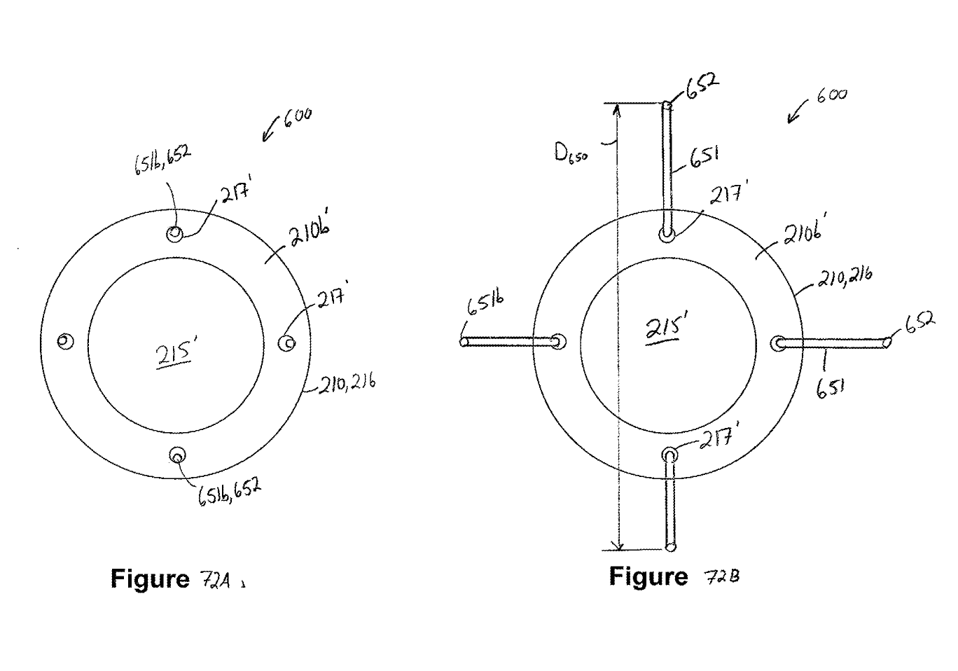

In the embodiment shown in FIGS. 64-66, expandable member 550 comprises resilient wires 551 extending from shaft 523 of stylet 520. However, in other embodiments, the stylet may be eliminated and the individual resilient wires of the expandable member may be slidingly disposed in elongate passages in the introducer housing. For example, referring now to FIGS. 70-72B, an embodiment of an introducer 600 is shown. Introducer 600 comprises an outer housing 210′, a plurality of resilient wires 651 that form an expandable member 650, and an enlarged actuation head 621. Housing 210′ is substantially the same as housing 210 previously described. Namely, housing 210′ extends axially between a first or proximal end 210a′ and a second or distal end 210b′. In addition, housing 210′ includes a base 211 as previously described at proximal end 210a′ and an elongate tubular or sleeve 216′ extending from base 211 to end 210b′. Sleeve 216′ has a length L_{216′} measured axially between base 211 and end 210b′ that is greater than vascular sheath length L₁₀₀. Base 211 has an outer diameter D₂₁₁, and sleeve 216′ has an outer diameter D₂₁₆ that is less than diameter D₂₁₁. Housing 210 also includes a central throughbore or passage 215′ that extends axially through base 211 and sleeve 216′ between ends 210a′, 210b′. Passage 215′ has a diameter D_{215′}. A guide wire such as wire 430 previously described may be slidingly disposed in passage 215′. However, in this embodiment, housing 210′ includes a plurality of circumferentially spaced throughbores 217′ extending axially between ends 210a′, 210b′. Further, in this embodiment, no stylet is provided. Rather, each resilient wire 651 has a first end 651a fixed to head 621 and a free end 651b comprising a blunted or rounded tip 652. Further, one wire 651 is slidingly disposed in each housing throughbore 217′. Thus, axial movement of head 621 moves wires 651 axially through bores 217′ of housing 210′. As shown in FIGS. 71, 72A, and 72B, the distal portions of each wire 651 define expandable member 650.

As with wires 551 previously described, in this embodiment, each wire 651 is biased to the expanded position shown in FIGS. 71 and 72B. In the radially expanded position, expandable member 650 has an outer diameter D₆₅₀ that is greater than outer diameter D_{216′} of housing sleeve 216′. However, in the collapsed, unexpanded position, expandable member 650 (and each wire 651) is disposed within one of throughbores 217′ and is restricted from expanding radially outward. Thus, expandable member 650 is controllably collapsed by pulling wires 651 into housing throughbores 217′ at end 210b′, and is expanded by pushing wires 651 out of housing throughbores 217′ at end 210b′.

To facilitate such radial expansion and contraction, each wire 651 preferably comprises a biocompatible, resilient material capable of being repeatedly transitioned between the expanded and unexpanded positions. For example, the wires (e.g., wires 651) may be made of nitinol. As previously described, in this embodiment, expandable member 650 is biased to the radially expanded position shown in FIGS. 71 and 72B, and must be forced into the collapsed position by urging each wire 651 into one of housing throughbores 217′. In other words, expandable member 650 is relaxed in the radially expanded position, and unrelaxed and under radially compressive loads in the unexpanded position.

If expandable member 650 is pushed completely through housing throughbores 217′, expandable member 650 will transition to the expanded position shown in FIGS. 71 and 72B as throughbores 217′ will no longer prevent wires 651 from radially expanding. Thus, to maintain expandable member 650 in the collapsed position shown in FIG. 72A, each wire 651 should be maintained within one housing throughbore 217′. Once expandable member 650 (and wire 651) extends from housing 210′ and radially expands, it may be collapsed by simply withdrawing expandable member 650 (and wires 651) back into housing throughbores 217′ at end 210b′. Vascular sheath 100 and introducer 600 may be used in substantially the same manner as sheath 100 and introducer 500 previously described to enable controlled percutaneous access to both the arterial limb and the venous limb of an AV graft with a single puncture, while simultaneously offering the potential to reduce the likelihood of inadvertent loss of access to the AV graft.

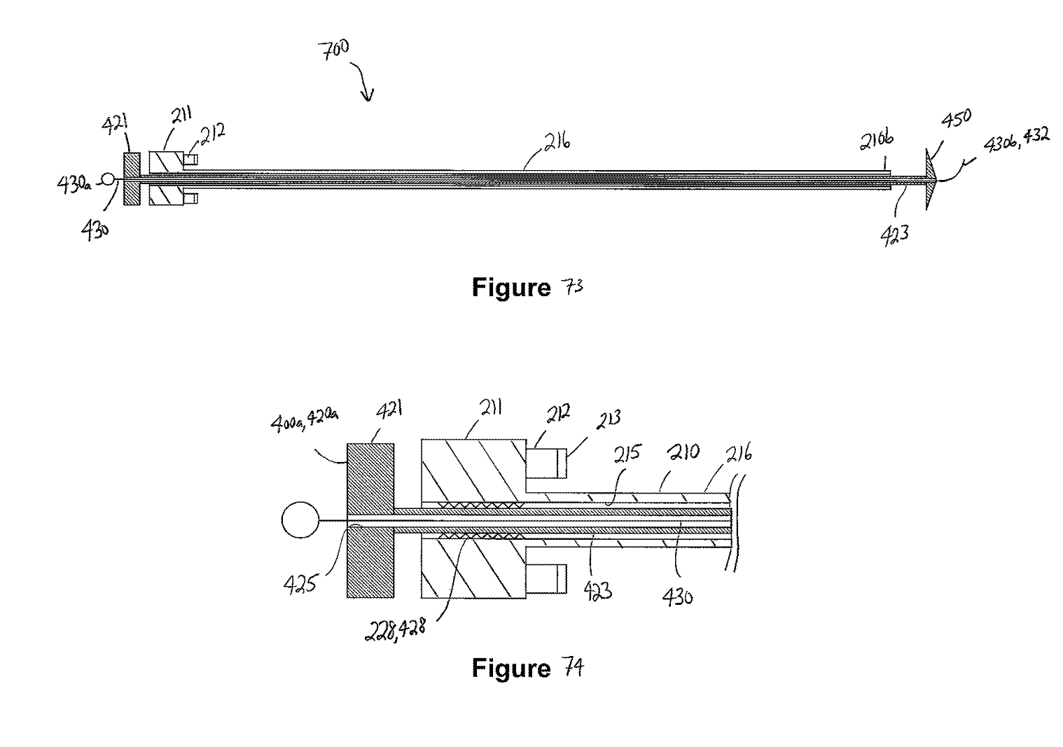

In the embodiments of introducers previously described (e.g., introducer 200, 300, 400, 500, 600), a distal expandable member (e.g., expandable member 250, 350, 450, 550, 650) is actuated by moving a proximal head (e.g., head 221, 321, 421, 521, 621) axially relative to a housing (e.g., housing 210). In particular, the operator urges the head axially towards the housing to radially expand the expandable member, and urges the head axially away from the housing to radially contract the expandable member. However, in other embodiments, actuation of the expandable member is achieved by rotation of a proximal head or handle, which is converted to axial motion by engagement of mating threads. For example, referring now to FIGS. 73 and 74, an embodiment of a vascular sheath introducer 700 is shown. In general, introducer 700 is a device used to position a vascular sheath, such as vascular sheath 100 previously described, into a vessel, AV graft, or AV fistula, and manipulate the vascular sheath within the vessel, AV graft, or AV fistula. In particular, introducer 700 enables a vascular sheath (e.g., vascular sheath 100) to be pivoted or swiveled back and forth within the lumen of an AV graft or fistula between the venous limb and arterial limb as many times as necessary, while reducing likelihood of inadvertent access loss and associated problems. As a result, embodiments of introducer 700 allow the operator to access both the arterial and venous limb of an AV graft or fistula via a single puncture. Further, embodiments of introducer 700 allow the operator to access an AV graft or AV fistula at any level and work in antegrade and retrograde fashions, via a single puncture.

Introducer 700 is substantially the same as introducer 400 previously described. Namely, introducer 700 includes a radially outer housing 210 as previously described and a radially inner stylet 420 as previously described. However, in this embodiment, stylet 420 includes external threads 428 that threadingly engage mating internal threads 228 disposed along housing throughbore 215. In this embodiment, threads 228 are disposed within base 211 of housing 210, and threads 428 are disposed along the outer surface of a portion of tubular 423 disposed within base 211. Due to the threaded engagement of stylet 420 and housing 210, stylet 420 is axially advanced through housing 210 to transition expandable member 450 between the expanded and unexpanded positions by rotating stylet 420 relative to housing 210. For example, the operator may rotate head 421 relative to base 211 in a first direction to transition expandable member 450 to the radially expanded position, and rotate head 421 relative to base 211 in the opposite direction to transition expandable member 450 to the unexpanded position.

Although stylet tubular 423 threadingly engages base 211 in this embodiment, in general, the introducer stylet (e.g., stylet 420) and the introducer housing (e.g., housing 210) may threadingly engage along any suitable axial location. For example, the stylet tubular or shaft may threadingly engage the housing base or the housing sleeve. Further, although threaded engagement is shown and described in connection with stylet tubular 423 and housing base 211, threaded engagement of the introducer stylet and the introducer housing may be employed in any of the embodiments described herein (e.g., introducer 200, 300, 400, 500, 600) to enable rotational actuation of the expandable member.

Embodiments of introducers described herein enable secured access to both the venous limb and arterial limb of an AV graft or fistula utilizing a single sheath and a single puncture. Specifically, embodiments of the introducer stabilize the vascular sheath within the lumen of the vessel or graft, allowing the sheath to be safely and easily swiveled into either limb of the dialysis access, as often as needed during the procedure, without losing access. As a result, embodiments described herein offer the potential for faster and easier procedures for access to the arterial and venous limbs of an AV graft or fistula with reduced pain and trauma to the patient, as well as reduced radiation exposure to the patient and staff. In addition, embodiments described herein offer the potential for decreased complications such as bleeding, pseudoaneurysm and infection, since only a single puncture is necessary. Such shorter procedures may also allow for cost reductions by decreasing procedural time and freeing up room/staff for other cases.

Although the puncture site (e.g., puncture 40) has been shown as being positioned at the apex of the AV graft (e.g., the apex of AV graft 20), in general, embodiments described herein allow the operator to puncture the native vessel or graft in almost any place, and is not limited to puncture sites at the apex of the AV graft or fistula. Thus, for example, the operator may place the sheath at any desired level of the AV graft or fistula, regardless of its configuration or shape. Moreover, in general, embodiments described herein may be employed to provide controlled and adjustable access to any vessel or lumen in a patient through a single puncture. It should also be appreciated that since embodiments described herein may be used with conventional vascular sheaths, operators do not have to change their current preferred techniques for treating failing dialysis accesses, while making the procedure better tolerated, faster, less expensive and safer for patient and staff. Although embodiments of methods and devices herein have been shown and described as enabling percutaneous access to both the arterial and venous limb of an AV graft, it should be appreciated that embodiments described herein may likewise be employed to provide percutaneous access to both the arterial and venous limb of an AV fistula with a single puncture.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the invention. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims.

What is claimed is:

1. An assembly for accessing a vessel, comprising:
an elongate housing having a longitudinal axis, a proximal end, and a distal end opposite the proximal end, wherein the housing includes a base extending from the proximal end, an elongated tubular extending from the distal end to the base, and a throughbore extending axially from the proximal end to the distal end;
a stylet extending axially through the throughbore of the housing, wherein the stylet has a proximal end comprising an actuation head external the throughbore of the housing, and a distal end opposite the proximal end of the stylet; and
an expandable member coupled to the distal end of the housing or the distal end of the stylet, wherein the expandable member has an expanded position with a first diameter and an unexpanded position with a second diameter that is less than the first diameter, wherein the stylet is adapted to transition the expandable member between the unexpanded position and the unexpanded position by moving axially through the housing.

2. The assembly of claim 1, wherein the stylet threadingly engages the housing; and
wherein the stylet is adapted to be rotated about the longitudinal axis relative to the housing in a first direction to transition the expandable member to the expanded position and rotated about the longitudinal axis relative to the housing in a second direction to transition the expandable member to the unexpanded position.

3. The assembly of claim 1, further comprising a locking mechanism disposed between the housing and the stylet, wherein the locking mechanism is adapted to releasably lock the stylet relative to the housing.

4. The assembly of claim 1, wherein the expandable member comprises an annular wire mesh, a plurality of circumferentially spaced strips, a plurality of resilient wires, a flexible annular flange, or a flexible, hollow conical structure.

5. The assembly of claim 1, wherein the distal end of the stylet extends axially from the distal end of the housing, and wherein the expandable member is axially disposed between the distal end of the housing and the stylet, and wherein the expandable member extends axially between a first end attached to the distal end of the housing and a second end attached to stylet.

6. The assembly of claim 5, wherein the distal end of the stylet comprises a tapered curved tip.

7. The assembly of claim 5, wherein the expandable member is biased to the unexpanded position.

8. The assembly of claim 5, wherein the stylet comprises a throughbore extending axially from the proximal end of the stylet to the distal end of the stylet, and wherein a guide wire is slidably disposed in the throughbore of the stylet.

9. The assembly of claim 1, wherein the expandable member has a first end attached to the distal end of the housing a second end opposite the first end, and wherein the distal end of the stylet is disposed within the expandable member.

10. The assembly of claim 9, wherein the distal end of the stylet comprises an engagement member that engages an inner surface of the expandable member and is adapted to exert an axial force on the expandable member to transition the expandable member from the expanded position to the unexpanded position.

11. The assembly of claim 10, wherein the engagement member of the stylet is a ball and the elongate member of the stylet is a wire.

12. The assembly of claim 9, wherein the second end of the expandable member comprises a tapered curved tip.

13. The assembly of claim 10, wherein the expandable member is biased to the radially expanded position.

14. The assembly of claim 1, wherein the expandable member is attached to the distal end of the stylet, and wherein the expandable member is disposed within the housing in the unexpanded position and disposed external the housing in the expanded position.

15. The assembly of claim 14, wherein a throughbore extends axially from the proximal end of the stylet through the distal end of the stylet and the expandable member.

16. The assembly of claim 15, further comprising a guide wire disposed in the throughbore extending through the stylet, wherein the guide wire has a proximal end extending from the throughbore of the stylet at the proximal end of the stylet and a distal end extending through the expandable member, and wherein the guide wire is adapted to be moved axially relative to the stylet and the housing.

17. The assembly of claim 16, wherein the expandable member is biased to the expanded position, and wherein the expandable member is disposed within the housing in the unexpanded position and external the housing in the expanded position.

18. The assembly of claim 16, wherein the distal end of the stylet includes an annular recess adapted to receive a portion of the expandable member when the expandable member is in the unexpanded position.

19. The assembly of claim 1, wherein the expandable member comprises a plurality of circumferentially spaced wires;
wherein each wire has a first end coupled to the head and a second end opposite the first end; and
wherein the second end of each wire is blunted.

20. The assembly of claim 19, wherein the expandable member is biased to the expanded position; and
wherein the second end of each wire is disposed within the housing in the unexpanded position and is external the housing in the expanded position.

21. The assembly of claim 1, further comprising a vascular sheath having a longitudinal axis, a proximal end, and a distal end opposite the proximal end, wherein the sheath includes a base extending axially from the proximal end, a tubular extending from the distal end to the base, and a throughbore extending between the proximal end and the distal end;
wherein the tubular of the elongate housing is disposed in the throughbore of the vascular sheath.

22. The assembly of claim 21, wherein the base of the housing includes a plurality of circumferentially spaced coupling arms adapted to releasably engage an annular recess on a radially outer surface of the base of the sheath.

23. A method for accessing a first limb and a second limb of an AV graft or fistula of a patient, the method comprising:
(a) providing a vascular sheath having a distal end disposed in the first limb and a proximal end external the patient, wherein the vascular sheath extends through a single puncture in the patient;
(b) inserting an introducer into the vascular sheath and advancing the introducer through the vascular sheath until a distal end of the introducer is disposed within the first limb;
(c) radially expanding an expandable member of the introducer within the first limb;
(d) withdrawing the vascular sheath from the first limb through the single puncture with the introducer after (c);
(e) restricting the distal end of the introducer from exiting the AV graft or fistula with the expandable member during (d); and
(f) advancing the introducer and the sheath through the single puncture and into the second limb.

24. The method of claim 23, wherein (e) comprises bearing against the inner surface of the AV graft or fistula with the expandable member.

25. The method of claim 23, wherein the introducer comprises:
an elongate housing having a longitudinal axis, a proximal end, and a distal end opposite the proximal end, and a throughbore extending axially from the proximal end to the distal end;
a stylet extending axially through the throughbore of the housing, wherein the stylet has a proximal end comprising an actuation head external the throughbore of the housing and a distal end opposite the proximal end of the stylet; and
the expandable member coupled to the distal end of the housing or the distal end of the stylet;
wherein (c) comprises moving the stylet axially relative to the housing.

26. The method of claim 25, wherein the stylet threadingly engages the housing, and wherein (c) comprises rotating the stylet about the longitudinal axis relative to the housing.

27. The method of claim 25, further comprising:
(g) radially contracting the expandable member after (f); and
(h) withdrawing the introducer from the sheath.

28. The method of claim 27, wherein (c) comprises extending the expandable member axially from the distal end of the housing and the distal end of the sheath; and wherein (g) comprises withdrawing the expandable member axially into the sheath.

29. The method of claim 23, wherein the introducer comprises:
an elongate housing having a longitudinal axis, a proximal end, and a distal end opposite the proximal end, a central throughbore extending axially from the proximal end to the distal end, and a plurality of circumferentially spaced throughbores radially spaced from the central throughbore, wherein each of the circumferentially spaced throughbores extends axially from the proximal end to the distal end;
an actuation head external the throughbore of the housing;
a plurality of wires, wherein each wire has a proximal end coupled to the actuation head and a distal end opposite the proximal end, wherein each wire is slidingly disposed in one of the circumferentially spaced throughbores of the housing, and wherein the distal ends of the wires forms the expandable member;
wherein (c) comprises moving the wires axially relative to the housing.
